(19) United States
(12) Patent Application Publication
(10) Pub. No.: US 2023/0140027 A1
(43) Pub. Date: May 4, 2023

(54) CELL-CULTURING INSTRUMENT-MACHINING DEVICE

(71) Applicant: KATAOKA CORPORATION, Kyoto-shi, Kyoto (JP)

(72) Inventors: Tadao MORISHITA, Kyoto (JP); Junichi MATSUMOTO, Kyoto (JP)

(21) Appl. No.: 17/911,527

(22) PCT Filed: Mar. 12, 2021
(86) PCT No.: PCT/JP2021/010165

§ 371 Date: Sep. 14, 2022

(30) Foreign Application Priority Data
Mar. 14, 2020 (JP) 2020-044841

(51) Int. Cl. C12M 1/42; C12M 1/22; C12N 13/00

(52) U.S. Cl. CPC C12M 35/02; C12M 23/10; C12N 13/00

(57) ABSTRACT

The processing apparatus 200 of the present disclosure includes: a laser irradiation unit 21 capable of applying a laser to the photothermal conversion layer 13 of the cell culture tool 100 including the cell culture base layer 11 and the photothermal conversion layer 13; and a control unit 22 for controlling the laser irradiation unit 21. The control unit 22 includes a setting section 221 and an irradiation control section 222. The setting section 221 sets an irradiation region to be irradiated with the laser in the cell culture tool 100. The irradiation control section 222 controls the laser irradiation unit 21 based on the irradiation region such that the laser irradiation unit 21 applies the laser to a corresponding region of the photothermal conversion layer 13.

### TECHNICAL FIELD

The present invention relates to a processing apparatus for a cell culture tool.

### BACKGROUND ART

When a cultured cell mass is processed into a desired shape, the cultured cell mass is subjected to a treatment such as cutting the cell mass into a desired shape, or a cell culture tool is processed in advance such that the cultured cell mass has a desired shape (Patent Document 1).

### CITATION LIST

### Patent Literature

Patent Literature 1: JP H03(1991)-007576 A

### SUMMARY OF INVENTION

### Technical Problem

According to the production method of Patent Literature 1, the cell culture tool is processed to obtain the cultured cell mass in a desired shape by patterning the surface of the cell culture tool through photolithography. However, photolithography requires various manufacturing facilities including a photomask forming apparatus and an exposure apparatus. Accordingly, there has been a demand for a processing apparatus having a simple structure and capable of controlling the shape of cells in a cell culture tool.

In light of the foregoing, it is an object of the present invention to provide a processing apparatus for cell culture tools, capable of controlling a region to which cells can adhere in a cell culture tool including a cell culture base layer and a photothermal conversion layer.

### Solution to Problem

In order to achieve the above object, the present invention provides a processing apparatus for a cell culture tool (hereinafter also referred to simply as “processing apparatus”), including: a laser irradiation unit capable of applying a laser to a photothermal conversion layer of a cell culture tool including a cell culture base layer and the photothermal conversion layer; and a control unit for controlling the laser irradiation unit, wherein the control unit includes a setting section and an irradiation control section, the setting section sets an irradiation region to be irradiated with the laser in the cell culture tool, and the irradiation control section controls the laser irradiation unit based on the irradiation region such that the laser irradiation unit apples the laser to a corresponding region of the photothermal conversion layer.

### Advantageous Effects of Invention

The processing apparatus according to the present invention can control a region to which cells can adhere in a cell culture tool including a cell culture base layer and a photothermal conversion layer.

### BRIEF DESCRIPTION OF DRAWINGS

FIG. 1 shows schematic views showing an example of the structure of a culture tool according to a first embodiment. FIG. 1A is a schematic perspective view of the culture tool of the first embodiment. FIG. 1B is a schematic cross-sectional view of the culture tool of the first embodiment as viewed along arrows I-I in FIG. 1A. FIG. 1C is a plan view of the culture tool of the first embodiment.

FIG. 2 shows schematic views illustrating an example of a production method and a processing method of the culture tool of the first embodiment.

FIG. 3 is a perspective view showing the structure of a processing apparatus according to a second embodiment.

FIG. 4 shows schematic views showing the processing apparatus of the second embodiment. FIG. 4A is a block diagram showing an example of the structure of a control unit of the processing apparatus of the second embodiment. FIG. 4B is a block diagram showing the structure of a CPU in the control unit.

FIG. 5 is a flowchart illustrating the steps of a processing method executed by the control unit of the processing apparatus of the second embodiment.

FIG. 6 shows an example of the structure of a control unit of a processing apparatus according to a third embodiment.

FIG. 7 is a flowchart illustrating an example of processing executed by the control unit in the third embodiment.

FIG. 8 shows schematic views illustrating a method of setting an irradiation region in the third embodiment.

FIG. 9 shows schematic views showing an example of control of a laser irradiation unit by an irradiation control section in the third embodiment.

FIG. 10 shows schematic views showing the structure of a processing apparatus according to a fourth embodiment.

FIG. 11 is a flowchart illustrating an example of processing executed by the processing apparatus of the fourth embodiment.

FIG. 12 is a perspective view showing an example of a processing apparatus according to a fifth embodiment.

FIG. 13 is a perspective view showing an example of a first region in the processing apparatus of the fifth embodiment.

FIG. 14 is a cross-sectional view of the first region as viewed along arrows I-I in FIG. 12.

FIG. 15A is an exploded perspective view showing an example of a culture vessel placement portion in the processing apparatus of the first embodiment. FIG. 15B is a cross-sectional view as viewed along arrows III-III in FIG. 15A.

FIG. 16 is a perspective view showing an example of the first region and a circulator in a state where an outer wall of the first region is removed in the processing apparatus of the fifth embodiment.

FIG. 17 is a cross-sectional view showing an upper part of the first region and the circulator as viewed along arrows II-II in FIG. 12.

FIG. 18A is a perspective view showing an example of the structure of a second region in the processing apparatus of the fifth embodiment. FIG. 18B is a perspective view showing another example of the structure of the second region.

FIG. 19 is a block diagram showing an example of the structure of a control section of the processing apparatus of the fifth embodiment.

FIG. 20 is a perspective view showing another example of the processing apparatus of the fifth embodiment.

FIG. 21 shows schematic views illustrating a method of setting an irradiation region in the third embodiment.

FIG. 22 shows schematic views illustrating another method of setting an irradiation region in the third embodiment.

### DESCRIPTION OF EMBODIMENTS

In the present invention, “cells” means, for example, isolated cells, or a cell mass (spheroid), tissue, or an organ composed of cells. The cells may be, for example, cultured cells or cells isolated from a living body. The cell mass, tissue, or organ may be, for example, a cell mass, cell sheet, tissue, or organ produced from the cells, or may be a cell mass, tissue, or organ isolated from a living body. The cells are preferably cells that adhere in an extracellular matrix (ECM)-dependent manner.

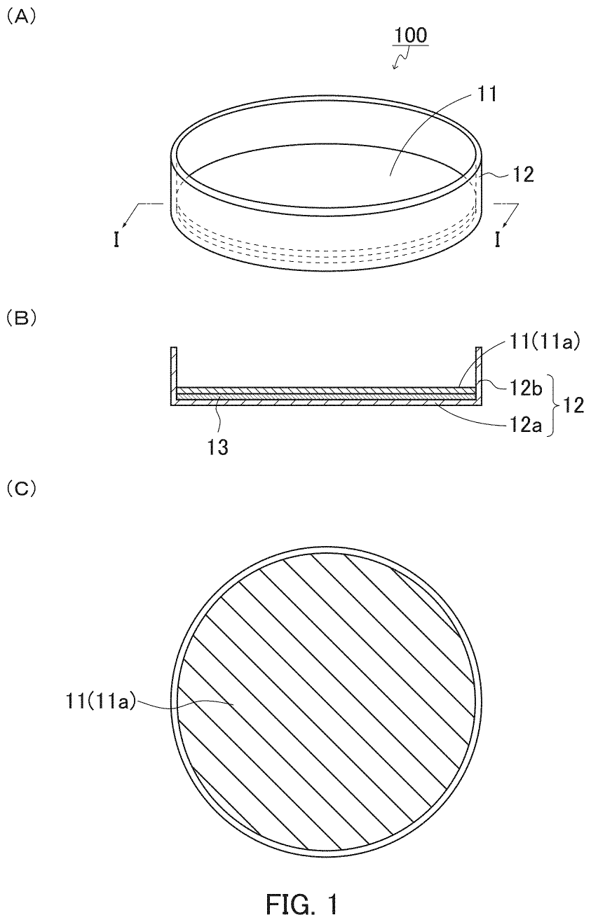

In the following, the processing apparatus according to the present invention and a cell culture tool to be processed by the processing apparatus will be described in details with reference to the drawings. It is to be noted, however, that the present invention is not limited by the following description. In FIGS. 1 to 22 to be described below, identical parts are given the same reference numerals, and duplicate explanations thereof may be omitted. In the drawings, the structure of each part may be shown in a simplified form as appropriate for the sake of convenience in explanation, and each part may be shown schematically with a dimensional ratio and the like that are different from the actual dimensional ratio and the like.

### First Embodiment

The present embodiment relates to an example of a cell culture tool to be processed by the processing apparatus of the present invention, an example of a method of producing the cell culture tool, and an example of a method of processing the cell culture tool using the processing apparatus of the present invention. FIG. 1 shows schematic views showing the structure of a culture tool 100 of the first embodiment. FIG. 1A is a schematic perspective view of the culture tool 100. FIG. 1B is a schematic cross-sectional view of the culture tool 100 as viewed along arrows I-I in FIG. 1A. FIG. 1C is a plan view of the culture tool 100. As shown in FIG. 1, the culture tool 100 includes a cell culture base layer 11, a photothermal conversion layer 13, and a vessel 12, which is a cell culture tool. In the present embodiment, the cell culture base layer 11 includes a cell adhesion region 11a to which cells can adhere. The vessel 12 has a bottom surface 12a and a side wall 12b. The cell culture base layer 11 is stacked on the bottom surface 12a. The photothermal conversion layer 13 is arranged between the cell culture base layer 11 and the bottom surface 12a. In other words, the photothermal conversion layer 13 and the cell culture base layer 11 are stacked on the bottom surface 12a in this order. As will be described below, in the present embodiment, a cell adhesion inhibitory region in which adhesion of cells is inhibited is formed by irradiating the culture tool 100 with light (laser) to change the adhesiveness of a cell culture base in the cell culture base layer 11. The culture tool 100 is used for cell culture after the cell adhesion inhibitory region is formed. Accordingly, the culture tool 100 can also be referred to as a culture tool before cell culture or a culture tool not yet provided with a layer of cells.

The cell culture base layer 11 is a layer containing a cell culture base. The cell culture base means, for example, a substance that serves as a scaffold of cells during cell culture. The cell culture base may be, for example, an extracellular matrix (ECM) or a substance that has a function as a scaffold for cells. Examples of the extracellular matrix include: elastin; entactin; collagens such as type I collagen, type II collagen, type III collagen, type IV collagen, type V collagen, and type VII collagen; tenascin; fibrillin; fibronectin; laminin; vitronectin; proteoglycans each composed of a sulfated glycosaminoglycan such as chondroitin sulfate, heparan sulfate, keratan sulfate, or dermatan sulfate and a core protein; glucosaminoglycans such as chondroitin sulfate, heparan sulfate, keratan sulfate, dermatan sulfate, and hyaluronic acid; Synthemax® (vitronectin derivative), and Matrigel® (a mixture of laminin, type IV collagen, heparin sulfate proteoglycan, entactin/nidogen, etc.). Of these, laminin is preferable. Examples of the laminin include laminin 111, laminin 121, laminin 211, laminin 213, laminin 222, laminin 311 (laminin 3A11), laminin 332 (laminin 3A32), laminin 321 (laminin 3A21), laminin 3B32, laminin 411, laminin 421, laminin 423, laminin 521, laminin 522, and laminin 523. The three numbers in each laminin indicate, from the first to the last, the names of the constituent subunits of the α, β, and γ chains, respectively. As a specific example, laminin 111 is composed of α1, β1, and γ1 chains. The laminin 3A11 is composed of α3A, β1, and γ1 chains. Examples of the cell culture base may further include peptide fragments of the above-described proteins and fragments of the above-described sugar chains. Specifically, examples of the peptide fragments of the proteins include fragments of laminins. Examples of the fragment of laminins include fragments of the above-described laminins, and specific examples thereof include laminin 211-E8, laminin 311-E8, laminin 411-E8, and laminin 511-E8. The laminin 211-E8 is composed of fragments of the α2, β1, and γ1 chains of laminin. The laminin 311-E8 is composed of fragments of the α3, β1, and γ1 chains of laminin. The laminin 411-E8 is composed of fragments of the α4, β1, and γ1 chains of laminin. The laminin 511-E8 is composed of, for example, fragments of the α5, β1, and γ1 chains of laminin.

The cell culture base can be denatured indirectly by applying light (laser) to the photothermal conversion layer 13, as will be described below. Specifically, the applied light is converted into heat, and the structure of the cell culture base is changed by the thus-generated thermal energy, thereby causing the indirect denaturation. In other words, the cell culture base is denatured by heat generated through the above-described light irradiation.

Although the culture tool 100 of the present embodiment includes one cell culture base layer 11, the culture tool 100 may include two or more cell culture base layers 11.

The cell culture base layer 11 may contain other components in addition to the cell culture base. Examples of the other components include buffers, salts, growth factors (cell growth factors), cytokines, and hormones.

In the culture tool 100 of the present embodiment, the cell culture base layer 11 is arranged (formed) only on the upper surface of the photothermal conversion layer 13. However, the present invention is not limited thereto. The cell culture base layer 11 need only be arranged in a region that allows contact with cells, for example, and may be arranged on an inner peripheral surface of the side wall 12b instead of or in addition to the upper surface of the photothermal conversion layer 13 in the culture tool 100. The cell culture base layer 11 may be formed on part or the whole of the region that allows contact with the cells. In the former case, the cell culture base layer 11 is preferably formed on the photothermal conversion layer 13 of the vessel 12 at the time of culturing cells.

The cell adhesion region 11a is a region to which the cells can adhere in the cell culture base layer 11. The cell culture base is adherable to the cells in, for example, an undenatured state. Accordingly, the cell adhesion region 11a can also be referred to as a region containing the cell culture base in an undenatured state, i.e., a region containing the undenatured cell culture base. Part or the whole of the cell culture base contained in the cell adhesion region 11a is in an undenatured state. When part of the cell culture base is in an undenatured state, for example, at least 50%, at least 60%, at least 70%, at least 75%, at least 80%, at least 85%, at least 90%, at least 95%, at least 96%, at least 97%, at least 98%, or at least 99% of the cell culture base in the cell adhesion region 11a is in an undenatured state. The proportion of the cell culture base in an undenatured state or a denatured state in the cell culture base can be determined by, for example, collecting the cell adhesion region 11a, performing native polyacrylamide gel electrophoresis (native PAGE) on the thus-collected material, and determining the proportion based on a change in the band position. The cell adhesion region 11a can also be referred to as, for example, a region that has not been irradiated with light in the production method to be described below. In the present embodiment, the cell culture base is denatured indirectly through light irradiation, whereby the adhesion capacity thereof with the cells is deteriorated. Thus, the cell adhesion region 11a contains the cell culture base in an undenatured state. It is to be noted, however, that the present invention is not limited thereto, and the cell culture base may be such that the adhesion with cells is inhibited when it is in an undenatured state and the adhesion capacity thereof with cells is improved when it is denatured either directly or indirectly through light irradiation. In this case, the cell adhesion region 11a contains the cell culture base in a denatured state. The cell culture base becomes adherable to cells when it is denatured indirectly through light irradiation.

The vessel 12 can be used to culture cells. In the vessel 12, a space surrounded by the bottom surface 12a and the side wall 12b is a region where cells can be cultured (cell culture region), and may also be referred to as a well, for example. The vessel 12 may be a cell culture vessel, and specific examples thereof includes substrates, dishes, plates, and flasks (cell culture flasks). The size, volume, and material of the vessel 12, the presence or absence of an adhesion treatment, and the like can be determined as appropriate according to the type and amount of cells to be cultured in the culture tool 100. The bottom surface 12a may be flat or substantially flat, and also may be a rough surface. Although the vessel 12 has the side wall 12b in the present embodiment, the side wall 12b may or may not be present. When the vessel 12 does not have the side wall 12b, the vessel 12 can also be referred to as, for example, a substrate or a support.

The material of the vessel 12 is not limited to particular materials, and may be, for example, a material that transmits a laser applied by a laser irradiation unit to be described below. Specific examples of such a material include plastic and glass that transmit a laser. Examples of the plastic include polystyrene polymers, acrylic polymers (such as polymethyl methacrylate (PMMA)), polyvinylpyridine polymers (such as poly(4-vinylpyridine) and 4-vinylpyridine-styrene copolymers), silicone polymers (such as polydimethylsiloxane), polyolefin polymers (such as polyethylene, polypropylene, and polymethylpentene), polyester polymers (such as polyethylene terephthalate (PET) and polyethylene naphthalate (PEN)), polycarbonate polymers, and epoxy polymers.

Although the vessel 12 has one cell culture region, the vessel 12 may have a plurality of cell culture regions. In the latter case, it can also be said that the vessel 12 has a plurality of wells, for example. Also, in the latter case, the cell culture base layer 11 and the photothermal conversion layer 13 may be formed in any one of the cell culture regions, in some of the cell culture regions, or in all the cell culture regions. In other words, in the vessel 12, the cell culture base layer 11 and the photothermal conversion layer 13 may be formed in any one or more of the wells or in all of the wells.

In the present embodiment, the vessel 12 may include a lid. The lid can cover the top of the vessel 12 in a detachable manner, for example. The lid is placed so as to face the bottom surface 12a, for example. The lid may be, for example, a lid of the cell culture vessel.

The photothermal conversion layer 13 is a layer capable of converting light into heat. The photothermal conversion layer 13 contains, for example, molecules capable of converting light into heat (photothermal conversion molecules). Preferably, the photothermal conversion molecules are composed of, for example, a polymer (macromolecules) containing a dye structure (chromophore) that absorbs light L having a wavelength used for irradiation in a processing method for the culture vessel 100 to be described below. Preferably, the photothermal conversion molecules can be easily coated onto the vessel 12. Examples of the dye structure that absorbs light L include derivatives of organic compounds, such as azobenzene, diarylethene, spiropyran, spirooxazine, fulgide, leuco dyes, indigo, carotinoid (such as carotene), flavonoid (such as anthocyanin), and quinoid (such as anthraquinone). Examples of the skeleton constituting the polymer include acrylic polymers, polystyrene polymers, polyolefin polymers, polyvinyl acetate and polyvinyl chloride, polyolefin polymers, polycarbonate polymers, and epoxy polymers. As a specific example, the photothermal conversion molecule may be, for example, poly[methylmethacrylate-co-(disperse yellow-7-methacrylate)]((C₅H₈O₂)ₘ(C₂₃H₂₀N₄O₂)ₙ) represented by the following formula (1). In the following formula (1), as the structure of azobenzene in the polymer, not only unsubstituted azobenzene but also any of various structures modified with a nitro group, an amino group, a methyl group, or the like may be employed. In the following formula (1), m and n each represent a mole percentage. The sum of m and n is, for example, 100 mol %. m and n may be the same or different from each other, for example. The photothermal conversion layer 13 may contain, for example, one type of photothermal conversion molecules or two or more types of photothermal conversion molecules.

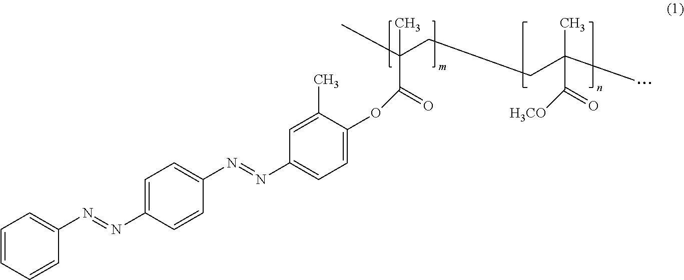

Although one photothermal conversion layer 13 is provided in the culture tool 100 of the first embodiment, a plurality of photothermal conversion layers 13 may be provided. In this case, it is preferable to arrange the plurality of photothermal conversion layers 13 between the cell culture base layer 11 and the bottom surface 12a. Although the photothermal conversion layer 13 is arranged in contact with the cell culture base layer 11 in the culture tool 100 of the present embodiment, it may be arranged so as not to be in contact with the cell culture base layer 11. In this case, the photothermal conversion layer 13 and the cell culture base layer 11 may be thermally connected to each other. Specifically, a heat conductive layer for transferring heat generated in the photothermal conversion layer 13 to the cell culture base layer 11 is formed between the photothermal conversion layer 13 and the cell culture base layer 11. The heat conductive layer contains molecules with high thermal conductivity, such as molecules of a metal, for example.

The photothermal conversion layer 13 may contain other components in addition to the above-described photothermal conversion molecules. The other components may be, for example, a polymer curing agent and unpolymerized monomers.

Although the photothermal conversion layer 13 is present only on the upper surface of the bottom surface 12a in the culture tool 100 of the present embodiment, the present invention is not limited thereto. The photothermal conversion layer 13 need only be arranged adjacent to the cell culture base layer 11, for example, and may be formed inside the vessel 12, for example. In this case, the photothermal conversion layer 13 is preferably formed on the upper surface of the bottom surface 12a of the vessel 12.

Although the photothermal conversion layer 13 is present on the entire upper surface of the bottom surface 12a in the culture tool 100 of the present embodiment, the present invention is not limited thereto. The photothermal conversion layer 13 may be formed on a portion of the bottom surface 12a, for example.

Although the photothermal conversion layer 13 is present only on the upper surface of the bottom surface 12a in the culture tool 100 of the present embodiment, the present invention is not limited thereto. The photothermal conversion layer 13 need only be arranged so as to be thermally connected to the cell culture base layer 11, for example, and may be arranged on the inner peripheral surface of the side wall 12b instead of or in addition to the upper surface of the bottom surface 12a in the culture tool 100. Also, the photothermal conversion layer 13 may be formed so as to be thermally connected to part or the whole of the cell culture base layer 11. In the former case, the photothermal conversion layer 13 is preferably formed on the bottom surface 12a of the vessel 12 at the time of culturing cells.

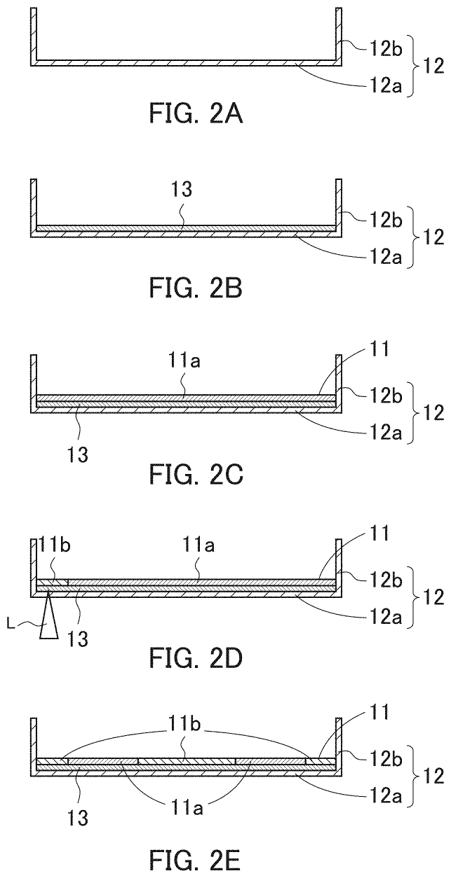

Next, a method for producing the culture tool 100 (the production method according to the present embodiment) and a processing method for controlling a region to which cells can adhere in the culture tool 100 (the processing method according to the present embodiment) will be described with reference to FIG. 2. FIG. 2 shows schematic views illustrating an example of the production method and the processing method of the culture tool 100. In the production method of the culture tool 100 of the present embodiment, using a cell culture base in an undenatured state, a cell culture base layer 11 is formed on a photothermal conversion layer 13. Then, in the processing method according to the present embodiment, the photothermal conversion layer 13 is irradiated with light, whereby the light is converted into heat by the photothermal conversion layer 13. Thus, in the production method according to the present embodiment, by heat generated in the photothermal conversion layer 13, the cell culture base in the cell culture base layer 11 adjacent to a region where the heat is generated is denatured, whereby a cell adhesion inhibitory region 11b is formed.

In the production method of the present embodiment, first, a vessel 12 is prepared, as shown in FIG. 2A (preparation step). The vessel 12 may be purchased commercially or prepared in-house, as described above.

Next, in the production method of the present embodiment, a photothermal conversion layer 13 containing the above-described photothermal conversion molecules is formed on a bottom surface 12a of the vessel 12, as shown in FIG. 2B (conversion layer forming step). The photothermal conversion layer 13 can be formed by, for example, a known film formation method, and specific examples of the method include coating, printing (screening), vapor deposition, sputtering, casting, and spin coating. Specifically, the photothermal conversion layer 13 can be formed, for example, by introducing, through spin coating, casting, or the like, a raw material solution containing the dye structure-containing polymer described above or a raw material solution obtained by dissolving the dye structure-containing polymer in a solvent into the vessel 12, more specifically such that the raw material solution is in contact with the bottom surface 12a of the vessel 12, and then curing the raw material solution. Examples of the solvent include organic solvents such as 1,2-dichloroethane and methanol.

Next, in the production method of the present embodiment, a cell culture base layer 11 containing the cell culture base described above is formed on the photothermal conversion layer 13, as shown in FIG. 2C (base layer forming step). In this manner, the vessel 12 provided with the cell culture base layer 11 and the photothermal conversion layer 13 can be prepared by the production method of the present embodiment. In the production method of the present embodiment, the cell culture base used for forming the cell culture base layer 11 is in an undenatured state. The cell culture base is adherable to cells when it is in an undenatured state. Accordingly, as shown in FIG. 2C, the formed cell culture base layer 11 consists of a cell adhesion region 11a. The cell culture base layer 11 can be formed by, for example, a known film formation method, and specific examples thereof include coating, printing (screening), vapor deposition, sputtering, casting, and spin coating. In the case where the cell culture base is a biopolymer such as a protein, the cell culture base layer 11 is preferably formed by coating, which can inhibit denaturation of the cell culture base. In this case, the cell culture base layer 11 may be formed by, for example, introducing a solvent containing an undenatured cell culture base into the vessel 12 and then allowing the vessel 12 to stand still. The solvent is, for example, an aqueous solvent and preferably water. The time period for which the vessel 12 is allowed to stand still is, for example, 30 minutes to one day. The temperature at which the vessel 12 is allowed to stand still is, for example, 4° C. to 40° C. In the case where the cell culture base is laminin 511-E8 and the coating concentration is 0.5 μg/cm², the vessel 12 is allowed to stand still for at least one hour at a temperature of about 37° C. (35° C. to 39° C.), for example. In the base layer forming step, after the vessel 12 is allowed to stand still as described above, the solvent containing the undenatured cell culture base is removed. After the removal of the solvent, the inside of the vessel 12 may be washed with a solvent that does not contain the cell culture base.

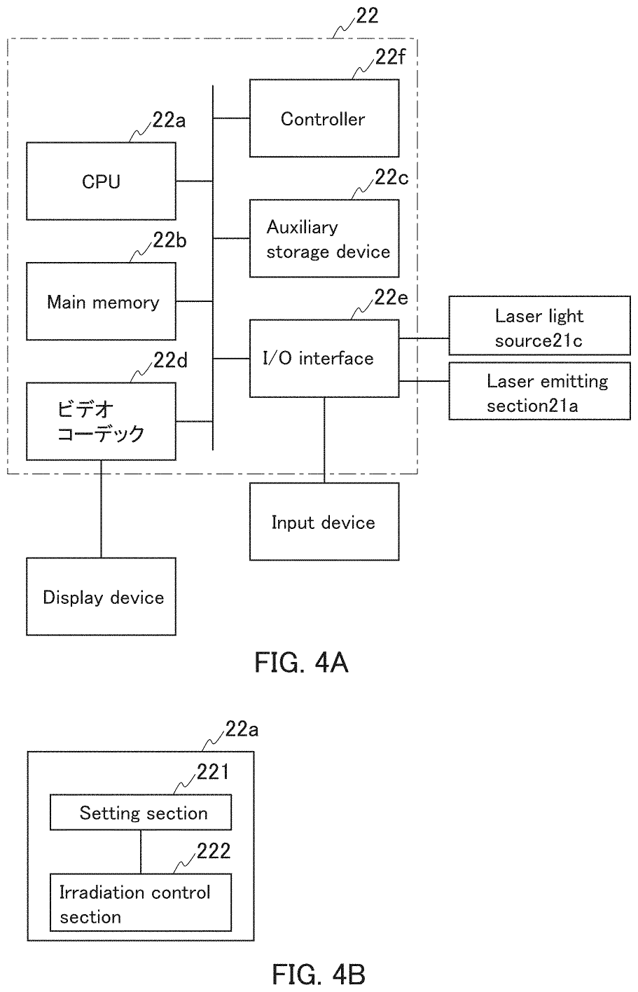

Next, in the culture tool 100 of the present embodiment, a region to which cells can adhere is demarcated through light (laser) irradiation. The light irradiation is performed using a processing apparatus according to the present invention to be described below. In the processing method of the present embodiment, as shown in FIG. 2D, the vessel 12 (cell culture tool) provided with the cell culture base layer 11 and the photothermal conversion layer 13 is irradiated with light L to denature the cell culture base, whereby a cell adhesion inhibitory region 11b is formed (inhibitory region forming step). Specifically, in the inhibitory region forming step, the photothermal conversion layer 13 is irradiated with light L. More specifically, the photothermal conversion layer 13 is irradiated with light L with the light L being focused on the photothermal conversion layer 13. As described above, the photothermal conversion layer 13 contains photothermal conversion molecules that convert light into heat. Accordingly, the photothermal conversion layer 13 irradiated with light L converts light energy of the light L into thermal energy. As a result, the temperature of a region of the photothermal conversion layer 13 irradiated with the light L increases, and this in turn increases the temperature of a region of the cell culture base layer 11 adjacent to the region irradiated with the light L, thereby changing the structure of the cell culture base in the cell culture base layer 11. In the inhibitory region forming step, the cell culture base is denatured in this manner, whereby a cell adhesion inhibitory region 11b is formed. The light L is preferably controlled so as to be focused on the photothermal conversion layer 13. In the inhibitory region forming step, it is preferable that the solvent is present on the cell culture base layer 11. In the production method of the present embodiment, the cell culture base is adherable to the cells when it is in an undenatured state, for example. Accordingly, the light L is applied to a region of the photothermal conversion layer 13 corresponding (adjacent) to a region for forming the cell adhesion inhibitory region 11b. More specifically, in FIG. 4D, the light L is applied to the corresponding region of the photothermal conversion layer 13, present immediately below the region for forming the cell adhesion inhibitory region 11b.

The wavelength of the light L can be set as appropriate according to the absorption wavelength of the photothermal conversion molecules contained in the photothermal conversion layer 13. The light L may have a wavelength of, for example, ultraviolet light, visible light, or infrared light. As a specific example, in the case of the polymer represented by the formula (1), the wavelength of the light L is 390 to 420 nm, for example. The light is preferably a laser beam because it allows the cell adhesion inhibitory region 11b to be formed precisely. The spot diameter (beam width) of the light L can be set as appropriate according to the amount of energy of the light L, for example. When the light L has a relatively small amount of energy, the spot diameter is set relatively small. When the light L has a relatively large amount of energy, the spot diameter is set relatively large. The spot diameter of the light L is, for example, 10 to 200 m. The amount of energy (output power) of the light L is, for example, the amount of energy to denature the cell culture base of the cell culture base layer 11 present in a region corresponding to (adjacent to) a portion irradiated with the light L in the photothermal conversion layer 13, and can be set as appropriate according to the type of the cell culture base and the type of the photothermal conversion molecules. The amount of energy of the light L is preferably the amount of energy to achieve a temperature at which cells in a cell layer stacked on the cell culture base layer 11 die. As a specific example, the amount of energy of the light L is the amount of energy to increase the temperature of the cell culture base in the cell culture base layer 11 at a portion irradiated with the light L to 50° C. or more, 60° C. or more, 70° C. or more, 80° C. or more, or 90° C. or more, and preferably to 100° C. or more, 110° C. or more, or 120° C. or more. The upper limit of the temperature is, for example, 200° C. In the inhibitory region forming step, the light L may be applied, for example, to increase the temperature of the photothermal conversion layer 13 so as to cause the cell culture base to have a temperature given above as examples. The scanning speed of the light L can be set as appropriate according to the spot diameter and the amount of energy of the light L, for example. When the amount of light energy per unit area of the spot diameter is relatively low, the scanning speed of the light L is set relatively low. When the amount of energy light per unit area of the spot diameter is relatively high, the scanning speed of the light L is set relatively high. As a specific example, the scanning speed of the light L is, for example, 100 mm/sec or less. The amount of energy of the light L is about 0.5 W (0.3 to 0.7 W) when the light L is a visible-light laser (405 nm), the spot diameter is 45 m, and the scanning speed of the light L is 80 mm/sec.

The cell adhesion inhibitory region 11b is a region where the adhesion of cells is inhibited. As described above, the cell culture base is adherable to cells when, for example, it is in an undenatured state. Thus, the cell adhesion inhibitory region 11b can also be referred to as, for example, a region containing the cell culture base in a denatured state, i.e., a region containing a thermally denatured product of the cell culture base. Part or the whole of the cell culture base contained in the cell adhesion inhibitory region 11b is in a denatured state. When part of the cell culture base is in a denatured state, for example, at least 50%, at least 60%, at least 70%, at least 75%, at least 80%, at least 85%, at least 90%, at least 95%, at least 96%, at least 97%, at least 98%, or at least 99% of the cell culture base in the cell adhesion inhibitory region 11b is in a denatured state. The cell adhesion inhibitory region 11b also can be referred to as, for example, a region that has been irradiated with the light L. The cell adhesion inhibitory region 11b is a region where adhesiveness to cells is deteriorated as compared to that in the cell adhesion region 11a, for example. Specifically, for example, the number of cells adhering to the cell adhesion inhibitory region 11b per unit area is reduced by at least 30%, at least 40%, at least 50%, at least 60%, at least 70%, at least 75%, at least 80%, at least 85%, at least 90%, at least 95%, at least 96%, at least 97%, at least 98%, or at least 99% and preferably by 100% as compared to the number of cells adhering to the cell adhesion regions 11a per unit area. The numbers of adhering cells per unit area in the respective regions are determined by, for example, tests conducted under the same conditions except for the state of the cell culture base. In the tests, inducted pluripotent stem cells (iPS cells) are preferably used. In this case, culture conditions used in the tests are conditions under which the iPS cells remain undifferentiated. In the present embodiment, the cell culture base is denatured indirectly through light irradiation, whereby the adhesion capacity thereof with cells is deteriorated. Thus, the cell adhesion inhibitory region 11b contains the cell culture base in a denatured state. It is to be noted, however, that the present invention is not limited thereto, and the cell culture base may be such that the adhesion with cells is inhibited when it is in an undenatured state and the adhesion capacity thereof with cells is improved when it is denatured indirectly through light irradiation. In this case, the cell adhesion inhibitory region 11b contains the cell culture base in an undenatured state. Also, the cell culture base inhibits the cell adhesion in the absence of light irradiation.

Then, in the processing method according to the present embodiment, the culture tool 100 including the cell adhesion region 11a and the cell adhesion inhibitory region 11b is produced, as shown in FIG. 2E. In the production method of the present embodiment, the cell culture base is adherable to cells when it is in an undenatured state. Accordingly, in the inhibitory region forming step, light L is applied to a region of the photothermal conversion layer 13 adjacent to a region for forming a cell adhesion inhibitory region 11b. It is to be noted, however, that the present invention is not limited thereto, and in the inhibitory region forming step, light L may be applied to a region of the photothermal conversion layer 13 adjacent to a region for forming a cell adhesion region 11a. In this case, the cell culture base is adherable to cells when it is in a denatured state.

In the present embodiment, light energy can be efficiently converted into thermal energy by using the photothermal conversion layer 13. Accordingly, in the present embodiment, the cell culture base in a region of the cell culture base layer 11 adjacent to the region of the photothermal conversion layer 13 irradiated with light L can be efficiently denatured. Thus, a processing apparatus according to the present invention to be described below can control a region to which cells can adhere in the culture tool 100 of the present embodiment.

### Second Embodiment

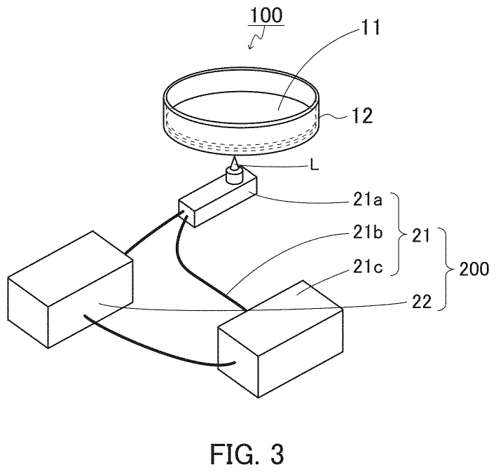

The present embodiment relates to an example of a processing apparatus. FIG. 3 shows an example of the structure of a processing apparatus according to the present embodiment. FIG. 3 is a perspective view showing an example of the structure of the processing apparatus of the present embodiment. As shown in FIG. 3, a processing apparatus 200 of the present embodiment includes a laser irradiation unit 21 and a control unit 22. The laser irradiation unit 21 includes a laser emission section 21a, an optical fiber 21b, and a laser source 21c. The control unit 22 is connected to the laser irradiation unit 21, more specifically, to the laser emission section 21a and the laser source 21c of the laser irradiation unit 21.

Although the laser irradiation unit 21 includes the laser emission section 21a, the optical fiber 21b, and the laser source 21c in the processing apparatus 200 of the present embodiment, the laser irradiation unit 21 is not limited thereto as long as it is capable of applying a laser to the photothermal conversion layer 13 of the culture tool 100. The laser irradiation unit 21 may be configured such that, for example, it includes the laser source 21c and applies a laser directly from the laser source 21c to the photothermal conversion layer 13 of the culture tool 100. In the case where a laser from the laser source 21c is guided to the laser emission section 21a, the laser may be guided using, instead of the optical fiber 21b, a light guide unit such as a mirror or a micro electro-mechanical system (MEMS). However, the optical fiber 21b is preferable because it allows the laser source 21c to be arranged at any position, can reduce the size of the processing apparatus 200, and also can reduce the weight of the processing apparatus 200 as compared with the case of using other light guide units.

In the processing apparatus 200 of the present embodiment, the laser emission section 21a is configured such that the irradiation position of a laser L emitted therefrom is movable by a laser moving unit (not shown). Alternatively, the laser irradiation unit 21 may be configured such that the irradiation position of the laser L is movable using a galvanometer mirror and an f6 lens, for example. Regarding the laser moving unit, reference can be made to the description thereon to be presented below.

The laser source 21c is, for example, a device that emits a continuous-wave laser or a pulsed laser. The laser source 21c may emit, for example, a high-frequency laser that has a long pulse width and approximates to a continuous wave. The output power of a laser emitted by the laser source 21c is not limited to particular values, and can be determined as appropriate according to, for example, the above-described absorption wavelength of the photothermal conversion molecules in the photothermal conversion layer 13. The wavelength of a laser emitted by the laser source 21c is not limited to particular values, and the laser may be, for example, a laser with a wavelength of 405 nm, 450 nm, 520 nm, 532 nm, or 808 nm, such as a visible-light laser or an infrared laser. As a specific example, the laser source 331 may be a continuous-wave diode laser having a maximum output power of 5 W and a wavelength in the vicinity of 405 nm.

The control unit 22 controls the laser irradiation unit 21. FIG. 4 shows block diagrams showing the hardware structure of the control unit 22. As shown in FIG. 4, the control unit 22 includes a central processing unit (CPU) 22a, a main memory 22b, an auxiliary storage device 22c, a video codec 22d, an I/O interface 22e, and other components, and they are controlled by a controller (a system controller, an I/O controller, or the like) 22f and operate in cooperation with each other. These components are connected to each other via busses, for example. Examples of the auxiliary storage device 22c include storage units such as a flash memory and a hard disk drive. The video codec 22d includes: a graphics processing unit (GPU) configured to generate a screen to be displayed based on a drawing instruction received from the CPU 22a and to transmit the screen signals to, for example, a display device or the like provided outside the processing apparatus 200; and a video memory for temporarily storing data concerning the screen and the image. The input-output (I/O) interface 22e is a device that is communicably connected to and controls the laser emission section 21a and the laser source 21c. The I/O interface 22e may include a servo driver (servo controller). The I/O interface 22e may be connected to, for example, an input device provided outside the processing apparatus 200. Examples of the display device include monitors that output images (e.g., various image display devices such as a liquid crystal display (LCD) and a cathode ray tube (CRT) display). Examples of the input device include pointing devices such as a touch panel, a trackpad, and a mouse, a keyboard, and a push button, each operable by an operator with his/her fingers. The auxiliary storage device 22c stores a program executed by the control unit 22. At the time of executing the program, the program is read into the main memory 22b and decoded by the CPU 22a. The control section 22 controls each component according to the program.

The CPU 22a operates in cooperation with other components via the controller 22f (such as a system controller or an I/O controller) and is responsible for overall control of the processing apparatus 200. In the control unit 22, the CPU 22a executes the above-described program and other programs, and also, read and write various types of information, for example. Specifically, as shown in FIG. 4B, the CPU 22a functions as, for example, a setting section 221 and an irradiation control section 222. Although the control unit 22 includes the CPU as an arithmetic unit, the control unit 22 may include another arithmetic unit such as a graphics processing unit (GPU) or an accelerated processing unit (APU), or may further include such a processing unit in combination with the CPU. The CPU 22a functions, for example, as respective sections, including an acquisition section and other sections, to be described below, and also, as respective sections other than a storage section in the third and fourth embodiments.

The main memory 22b is also referred to as a main storage device. When the CPU 22 executes processing, the main memory 22b reads in various operation programs, including the above-described program, stored in the auxiliary storage device 22c to be described below, for example. The CPU 22a then reads out data from the main memory 22b, decodes the data, and executes the programs. The main memory 22b is a random-access memory (RAM), for example. Examples of the main memory 22b further include a read-only memory (ROM).

The auxiliary storage device 22c stores operation programs including the above-described program. The auxiliary storage device 22c includes, for example, a storage medium and a drive for reading and writing with respect to the storage medium. The storage medium is not limited to particular types of storage media, and for example, may be either a built-in or external storage medium, and may be a hard disk (HD), a Floppy® disk (FD), CD-ROM, CD-R, CD-RW, MO, DVD, a flash memory, a memory card, or the like. The drive is not limited to particular types of drives. The auxiliary storage device 22c may be, for example, a hard disk drive (HDD) in which the storage medium and the drive are integrated. When the control unit 22 includes the storage section, for example, the auxiliary storage device 22c serves as the storage section.

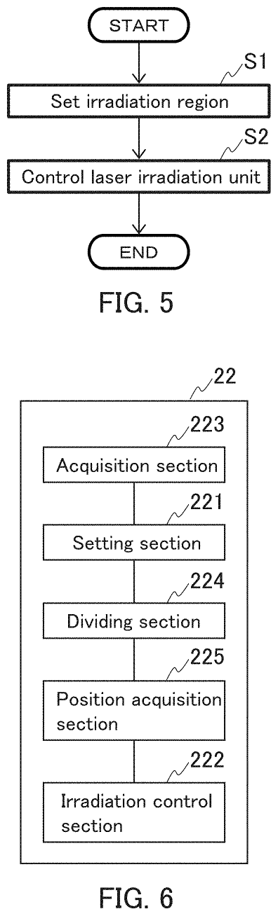

Next, a method for controlling the laser irradiation unit 21 by the control unit 22 of the processing apparatus 200 of the present embodiment will be described with reference to the flowchart of FIG. 5. FIG. 5 is a flowchart illustrating an example of the processing (S1 to S2) executed by the control unit 22.

First, in the step S1, the setting section 221 sets an irradiation region to be irradiated with a laser L in the culture tool 100 (setting step). Specifically, the setting section 221 associates each coordinate of the bottom surface 12a of the culture tool 100 with information on the presence or absence of irradiation of the laser L by the laser irradiation unit 21. The coordinates can be acquired by, for example, setting a coordinate plane in a plane including the bottom surface 12a. The coordinate plane can be set by, for example, setting an axis (X-axis) extending in one direction and an axis (Y-axis) extending in a direction orthogonal to the X-axis direction on the plane including the bottom surface 12a. The center position of the coordinate plane can be set, for example, inside the bottom surface 12a or outside the bottom surface 12a. The shape of the irradiation region set in the step S1 is not limited to particular shapes and can be any shape. Although the present embodiment describes an illustrative example in which the setting section 221 directly sets an irradiation region, the setting section 221 may set a non-irradiation region not to be irradiated with the laser L to set an irradiation region indirectly or may set both an irradiation region and a non-irradiation region.

The irradiation region may be set in advance or may be set during use of the processing apparatus 200, for example. In the case where the irradiation region is set in advance, information on the irradiation region (irradiation region information) is stored in the auxiliary storage device 22c, for example. Thus, the setting section 221 sets the irradiation region to be irradiated with the laser L using the irradiation region information stored in the auxiliary storage device 22c.

In the case where the irradiation shape is set during use of the processing apparatus 200, the control unit 22 may include, for example, an acquisition section for acquiring irradiation region information in which the irradiation region is specified. In this case, the control method of the present embodiment includes, prior to the step S1, the step of acquiring irradiation region information in which the irradiation region is specified by the acquisition section. Then, in the step S1, the setting section 221 sets the irradiation region to be irradiated with the laser L based on the irradiation region information.

The acquisition section can acquire the irradiation region information by acquiring information in which the irradiation region is not specified and then specifying the irradiation region using the information in which the irradiation region is not specified. Examples of the information in which the irradiation region is not specified include: an image showing the whole or part of the surface of the cell culture tool (image data); and information on the cell culture tool, such as the size, volume, and material of the cell culture tool, the presence or absence of an adhesion treatment, and the like (identification information).

When the information in which the irradiation region is not specified is an image showing the whole or part of the surface of the cell culture tool, the acquisition section may acquire the irradiation region information by identifying an indistinct region formed in the cell culture tool in the above-described image and setting such a region as the irradiation region. As a specific example, in the case where an indistinct region caused by the meniscus that occurs when acquiring the image of the cell culture tool is set as the irradiation region, the processing apparatus 200 can set the irradiation region in the following manner, for example. The meniscus means a curved liquid surface formed at the boundary between the cell culture tool and liquid introduced into the cell culture tool. The indistinct region means, for example, a region in which, owing to the curved liquid surface formed at the boundary between the cell culture tool and the liquid introduced into the cell culture tool, a decrease in the contrast or an increase in the luminance value from the boundary toward the center of the cell culture tool is caused. As a specific example, in the case where the image includes a phase-contrast image acquired by a phase-contrast microscope, the indistinct region means a region in the image captured in the state where, owing to the curved liquid surface formed at the boundary between the cell culture tool and the liquid introduced into the cell culture tool, a phase shift from the boundary toward the center of the cell culture tool is present.

First, the acquisition section acquires an image that includes the irradiation region or an image that may include the irradiation region. The image can be acquired by an optical observation device such as a phase-contrast microscope, for example. The image includes the whole or part of the surface of the cell culture tool and preferably includes the whole of the surface of the cell culture tool. Next, the acquisition section extracts the indistinct region from the image. Specifically, the acquisition section compares the luminance value of each pixel of the image and/or the contrast of each pixel present in a region of a predetermined size with the threshold value, and determines whether the pixel or the region is affected by the meniscus. Then, when the luminance value is lower than or equal to the threshold value and/or the contrast is higher than the threshold value, the acquisition section determines that the pixel or the region is not affected by the meniscus, i.e., the pixel or the region forms a distinct region. Subsequently, the acquisition section associates the distinct region with information that the laser L should not be applied or does not associate the distinct region with information that the laser L should be applied. On the other hand, when the luminance value is higher than the threshold value and/or the contrast is lower than or equal to the threshold value, the acquisition section determines that the pixel or the region is affected by the meniscus, i.e., the pixel or the region forms an indistinct region. Then, the acquisition section associates the indistinct region with information that the laser L should be applied or does not associate the indistinct region with information that the laser L should not be applied. In this manner, the acquisition section can acquire irradiation region information in which the irradiation region is specified. The threshold value may be, for example, specified by a user or set in advance using an image acquired by capturing an image of the cell culture tool to which liquid has been introduced.

When the information in which the irradiation region is not specified is an image showing the whole or part of the surface of the cell culture tool, the acquisition section may identify the cell culture tool in the image including the cell culture tool and may acquire irradiation region information in which the irradiation region is specified from information on the thus-identified cell culture tool. As a specific example, in the case where an indistinct region caused by the meniscus that occurs when acquiring an image of the cell culture tool is set as the irradiation region, the processing apparatus 200 can set the irradiation region in the following manner, for example. First, the acquisition section acquires an image including the cell culture tool. The image can be acquired by an optical observation device such as a phase-contrast microscope, for example. The image includes the whole or part of the surface of the cell culture tool and preferably includes the whole of the surface of the cell culture tool.

Next, the acquisition section extracts a region where the cell culture tool is present from the image. Specifically, the acquisition section identifies (specifies) from the image the type of the cell culture tool included therein. The method of identifying the cell culture tool may be extracting information on the cell culture tool, such as the size, thickness, and material of the cell culture tool, from the image and then matching the information against a database in which various types of cell culture tools are associated with information on each of the cell culture tools. The database may be provided outside the processing apparatus 200, or data may be stored in the auxiliary storage device 22c and used as a database. The above-described identification method may be performed by matching the image with images of various types of cell culture tools through image processing such as template matching. Then, the acquisition section identifies the cell culture tool specified by the above matching as the cell culture tool included in the image. Further, based on the thus-identified cell culture tool, the acquisition section extracts irradiation region information corresponding to the cell culture tool included in the image from a database in which various types of cell culture tools are associated with an irradiation region in which the irradiation region in each cell culture tool is specified. In the above-described manner, the acquisition section can acquire the irradiation region information from the image including the cell culture tool. Although the above example is directed to the case where the irradiation region information is acquired from an image including the cell culture tool using the features of the cell culture tool, the processing apparatus of the present invention is not limited thereto. When identification information (e.g., characters, graphics, and identifiers such as a QR Code®) that enables identification of the cell culture tool is provided (arranged) in the cell culture tool or a placement portion of the cell culture tool (e.g., a tool placement portion to be described below), the irradiation region information may be acquired using the identification information. In this case, the processing apparatus 200 further includes an identification information acquisition section for acquiring identification information of the cell culture tool, and the acquisition section can acquire irradiation region information associated with the cell culture tool based on the identification information of the cell culture tool. The identification information can be acquired, for example, using an optical observation device such as an optical microscope, as with the case of acquiring an image including the cell culture tool. The processing apparatus 200 may include a determination section for determining whether the image including the cell culture tool includes identification information. In this case, when the determination section determines that the image does not include identification information, the acquisition section acquires irradiation region information from the image including the cell culture tool. On the other hand, when the determination section determines that the image includes identification information, the identification information acquisition section acquires the identification information of the cell culture tool, and the acquisition section then acquires irradiation region information based on the identification information of the cell culture tool.

The irradiation region information may be, for example, an image in which the irradiation region is specified or information on a user-specified irradiation region. When the irradiation region is set using an image in which the irradiation region is specified, the setting section 221 can set the irradiation region by, for example, associating pixels satisfying a previously set condition in the image with information that the laser L should be applied and associating the remaining pixels with information that the laser L should not be applied. Alternatively, the setting section 221 may set the irradiation region by, for example, associating pixels satisfying a previously set condition in the image with information that the laser L should not be applied and associating the remaining pixels with information that the laser L should be applied. Also, the setting section 221 may set the irradiation region under conditions that are opposite to these conditions. The previously set condition may be, for example, a condition based on an irradiation region or a non-irradiation region in the image or a condition based on the contrast or luminance value of each pixel in the image. In this case, the setting section 221 can set the irradiation region by determining whether a region of interest is an irradiation region based on, for example, whether the contrast or luminance value of each pixel in the image satisfies the condition concerning the contrast or luminance value (e.g., the threshold value).

When the irradiation region is set using the information on a user-specified irradiation region, the setting section 221 can set the irradiation region by, for example, associating, in the above information, a region satisfying the previously set condition with information that the laser L should be applied and associating the remaining region with information that the laser L should not be applied. Alternatively, the setting section 221 may set the irradiation region by, for example, associating, in the above information, a region satisfying the previously set condition with information that the laser L should not be applied and associating the remaining region with information that the laser L should be applied. Also, the setting section 221 may set the irradiation region under conditions that are opposite to these conditions. The user specifies the irradiation region by, for example, enclosing the irradiation region. Thus, the previously set condition may be, for example, whether an enclosed region, i.e., a closed region, is formed in the user-specified irradiation region. In this case, the setting section 221 can set the irradiation region by determining whether a region of interest is the irradiation region based on, for example, whether there is a region forming a closed region in the information on the user-specified irradiation region.

Then, in the step S2, the irradiation control section 222 controls the laser irradiation unit 21 based on the irradiation region such that the laser irradiation unit 21 applies a laser to a region of the photothermal conversion layer 13 corresponding to the irradiation region in the culture tool 100. Examples of the control of the laser irradiation unit 21 by the irradiation control section 222 include controlling the irradiation position of the laser L in the photothermal conversion layer 13 and ON/OFF switching of irradiation of the laser L.

When the irradiation control section 222 controls the irradiation position of the laser, the irradiation control section 222 can control the irradiation position of the laser by, for example, controlling the start, stop, and/or speed of the movement of a moving unit that can move the laser irradiation unit 21. When the laser irradiation unit 21 includes a galvanometer mirror and an fo lens, the irradiation control section 222 can control the irradiation position of the laser by controlling the angle of the galvanometer mirror, for example.

When the irradiation control section 222 controls the ON/OFF of irradiation of the laser L, the irradiation control section 222 can control the ON/OFF of irradiation of the laser L by, for example, controlling the ON/OFF of laser light emission by the laser source 21c. The irradiation control section 222 controls the ON/OFF of laser light emission based on, for example, each coordinate of the bottom surface 12a in the irradiation region set by the setting section 221 and information on the presence or absence of irradiation of the laser L by the laser irradiation unit 21, associated with each coordinate.

The control unit 22 controls the irradiation of the photothermal conversion layer 13 of the culture tool 100 with the laser L by the laser irradiation unit 21 in the above-described manner, whereby the shape of a cell adhesion inhibitory region 11b formed on the cell culture base layer 11 of the culture tool 100 can be controlled. Although the processing apparatus 200 of the present embodiment is configured such that the control unit 22 is responsible for overall control of the processing apparatus 200, the processing apparatus of the present invention is not limited thereto and may be configured such that a control unit for the laser irradiation unit 21, such as a laser controller, is provided separately and the control unit for the laser irradiation unit 21 functions as the irradiation control section 222.

The processing apparatus 200 of the present embodiment can easily control a region to which cells can adhere in a cell culture tool including a cell culture base layer and a photothermal conversion layer by performing laser irradiation in a controlled manner.

### Third Embodiment

The present embodiment relates to another example of the processing apparatus. FIG. 6 shows an example of the structure of a control unit 22 of a processing apparatus according to the present embodiment. FIG. 6 is a block diagram showing an example of the structure of the control unit 22 of the processing apparatus of the present embodiment. As shown in FIG. 6, the control unit 22 of the processing apparatus of the present embodiment has the same structure as the control unit 22 of the processing apparatus of the second embodiment, except that it includes an acquisition section 223, a dividing section 224, and a position acquisition section 225 in addition to a setting section 221 and an irradiation control section 222, and the above description regarding the structure of the control unit 22 in the second embodiment also applies to the control unit 22 of the present embodiment. In the processing apparatus of the present embodiment, a CPU 22a functions as the setting section 221, the irradiation control section 222, the acquisition section 223, the dividing section 224, and the position acquisition section 225.

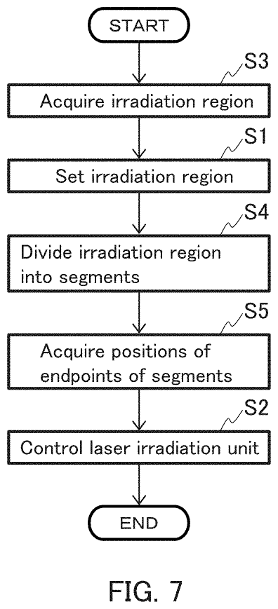

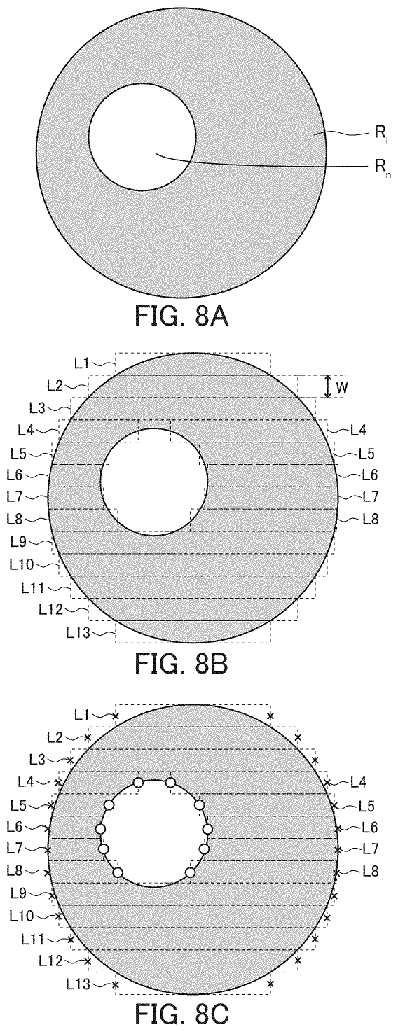

Next, a method for controlling a laser irradiation unit 21 by the control unit 22 of the processing apparatus of the present embodiment will be described using the flowchart of FIG. 7 with reference to an illustrative example in which, in a culture tool 100, a circular non-irradiation region (Rₙ) and the remaining irradiation region (Rᵢ) shown in FIG. 8A are subjected to laser irradiation by the processing apparatus of the present embodiment. FIG. 7 is a flowchart illustrating an example of processing (S1 to S5) executed by the control unit 22. FIG. 8 shows schematic views illustrating a method for setting the irradiation region.

First, in the step S3, the acquisition section 223 acquires irradiation region information in which the irradiation region is specified (acquisition step). Specifically, the acquisition section 223 acquires an image of a bottom surface 12a of the culture tool 100 as shown in FIG. 8A, including an irradiation region (Rᵢ) to be irradiated and non-irradiation region (Rₙ) not to be irradiated with a laser L by the laser irradiation unit 21. The image can be acquired by, for example, importing data including the image from the outside of the processing apparatus.

Next, in the step S1, the setting section 221 sets the irradiation region based on the image. Specifically, in the image acquired by the acquisition section 223, a region where the luminance value is either less than a predetermined value or greater than or equal to the predetermined value is set as the irradiation region Rᵢ. In the present embodiment, the predetermined value is, for example, a value at which the irradiation region Rᵢ shown in gray and the non-irradiation region Rₙ shown in white are distinguishable from each other. Accordingly, as shown in FIG. 8A, the setting section 221 sets the gray region as the irradiation region Rᵢ based on the predetermined value. As a result, the white region is indirectly set as the non-irradiation region Rₙ.

In the step S4, as shown in FIG. 8B, the dividing section 224 divides the thus-set irradiation region Rᵢ into segments based on an irradiation width W (processing width) of the laser. In the present embodiment, the dividing section 224 divides the irradiation region Rᵢ into strip-shaped segments with their longitudinal direction extending in the left-right direction (hereinafter also referred to as “X-axis direction”) in FIG. 8. As a result, in the step S4, the segments (L1 to L13) of the irradiation region are formed. The length of each of L1 to L13 in the upper-lower direction (hereinafter also referred to as “Y-axis direction”) corresponds to the irradiation width W. The irradiation width W also can be referred to as, for example, the length of the spot diameter of the laser irradiation unit 21 in the Y-axis direction. When the dividing section 224 divides a portion that extends over both the irradiation region Rᵢ and the non-irradiation region Rₙ, for example, when providing L4 to L8, the dividing section 224 divides the portion such that, of the resulting segments of the irradiation region, segments having the same coordinate in the Y-axis direction are considered as a single segment. Although the irradiation width W is set constant, the irradiation width W may vary. Although the width of the segments (L1 to L13) is set to be the same as the irradiation width W, it may be different from the irradiation width W. In the latter case, the width of the segments (L1 to L13) may be greater than the irradiation width W.

Next, in the step S5, the position information acquisition section 225 acquires the positions of the endpoints of the segments L1 to L13. Specifically, the position information acquisition section 225 acquires the coordinates of the endpoints at both ends of the segments, as indicated with cross marks (x) in FIG. 8C. The coordinates are, for example, coordinates on the XY plane that is set based on the X-axis direction and the Y-axis direction. For the segments including the non-irradiation region Rₙ, such as L4 to L8, the position information acquisition section 225 acquires the coordinates of the boundaries between the irradiation region Rᵢ and the non-irradiation region Rₙ as the laser ON/OFF switching positions, as indicated with circles (∘) in FIG. 8C. Then, the position information acquisition section 225 associates each of the segments L1 to 13 with the coordinates of the corresponding endpoints and the laser ON/OFF switching positions.

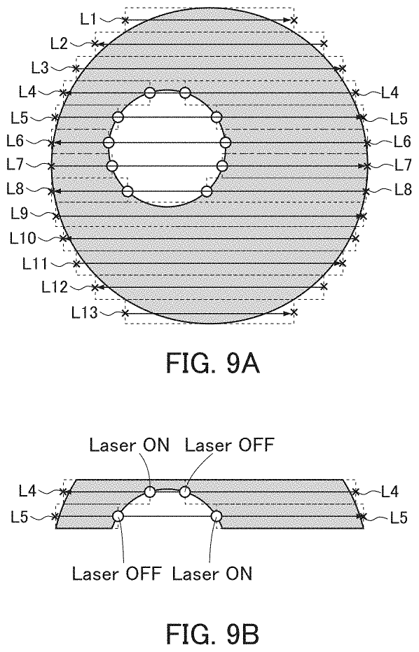

Next, control of the laser irradiation unit 21 by the irradiation control section 222 in the step S2 will be described with reference to FIG. 9. FIG. 9 shows schematic views showing an example of the control of the laser irradiation unit 21 by the irradiation control section 222. FIG. 9A is a schematic view showing an example of the control for the whole culture tool 100. FIG. 9B is a schematic view showing an example of the control for L4 and L5.

As shown in FIG. 9A, in the step S2, the irradiation control section 222 controls the laser irradiation unit 21 based on the segments L1 to L13 of the irradiation region and the coordinates of the corresponding endpoints and the laser ON/OFF switching positions such that the laser irradiation unit 21 applies the laser L to the corresponding regions of the photothermal conversion layer 13. Specifically, the irradiation control section 222 controls the laser irradiation unit 21 so as to apply the laser L from the left endpoint toward the right endpoint of L1. At this time, the irradiation control section 222 also acquires the irradiation position (coordinates) of the laser L from the laser irradiation unit 21. When irradiation of the laser L by the laser irradiation unit 21 is OFF, the irradiation control section 222 acquires, as the irradiation position of the laser L from the laser irradiation unit 21, a virtual irradiation position based on the assumption that the laser irradiation unit 21 applies the laser L. The virtual irradiation position can be calculated from the coordinates of the segment and the moving speed of the laser. The irradiation control section 222 may determine whether the position of the laser irradiation unit 21 aligns with the position of the segment of interest. In the processing apparatus of the present embodiment, the laser irradiation unit 21 is movable by a laser moving unit. Accordingly, the irradiation control section 222 can control the position of the laser irradiation unit 21 by controlling the position of the laser moving unit. For this reason, the irradiation control section 222 acquires the position of the laser moving unit as the irradiation position of the laser L from the laser irradiation unit 21. After the irradiation of L1 with the laser L is completed, the irradiation control section 222 controls the laser irradiation unit 21 such that the laser irradiation unit 21 can apply the laser L to the right endpoint of L2. Then, the irradiation control section 222 controls the laser irradiation unit 21 so as to apply the laser L to the corresponding region of the photothermal conversion layer 13 by moving the laser from the right endpoint toward the left endpoint of L2. Similarly, the irradiation control section 222 controls the laser irradiation unit 21 so as to apply the laser L from the left endpoint toward the right endpoint to L3.

Subsequently, the irradiation control section 222 controls the laser irradiation unit 21 so as to apply the laser L from the right endpoint toward the left endpoint of L4. L4 is set so as to extend over both the irradiation region Rᵢ and the non-irradiation region Rₙ. Thus, as shown in FIG. 9B, L4 includes two laser ON/OFF switching positions. As described above, the irradiation control section 222 has acquired the irradiation positions of the laser L from the laser irradiation unit 21. Accordingly, the irradiation control section 222 can control ON/OFF switching of irradiation of the laser L by the laser irradiation unit 21 based on whether the irradiation position of the laser L from the laser irradiation unit 21 coincides with the laser ON/OFF switching positions. Specifically, when the laser irradiation unit 21 is applying the laser L to a region that extends between the right endpoint and the circle on the right in L4, the irradiation control section 222 determines that the irradiation position of the laser L from the laser irradiation unit 21 does not coincide with the laser ON/OFF switching positions and thus does not control the laser ON/OFF switching by the laser irradiation unit 21. On the other hand, when the laser L from the laser irradiation unit 21 reaches the position of the circle on the right in L4, the irradiation control section 222 determines that the irradiation position of the laser L from the laser irradiation unit 21 coincides with the laser ON/OFF switching position and thus controls the laser ON/OFF switching by the laser irradiation unit 21. In this case, since the laser irradiation unit 21 is applying the laser L, the irradiation control section 222 controls the laser irradiation unit 21 so as to turn off the laser L. Further, when the laser L from the laser irradiation unit 21 reaches the position of the circle on the left in L4, the irradiation control section 222 determines that the irradiation position of the laser L from the laser irradiation unit 21 (the virtual irradiation position based on the assumption that the laser irradiation unit 21 applies the laser L) coincides with the laser ON/OFF switching position and thus controls the laser ON/OFF switching by the laser irradiation unit 21. In this case, since the laser irradiation unit 21 is not applying the laser L, the irradiation control section 222 controls the laser irradiation unit 21 so as to turn on the laser L. Then, the irradiation control section 222 controls the laser irradiation unit 21 such that the laser irradiation unit 21 can apply the laser L to the left endpoint of L4. The irradiation control section 222 controls the laser irradiation unit 21 such that regions of the photothermal conversion layer 13 corresponding to L5 to L13 are irradiated with the laser L in the same manner as in the above. Although the laser L is scanned over the respective segments sequentially and in alternating directions in the present embodiment, the scanning direction of the laser L in the processing apparatus of the present invention is not limited thereto and the laser may be scanned in one direction. Although L1 to L13 are irradiated with the laser L in this order in the present embodiment, there is no particular limitation on the order of irradiating the segments with the laser L.

The processing apparatus 200 of the present embodiment can easily control a region to which cells can adhere in a cell culture tool including a cell culture base layer and a photothermal conversion layer by performing laser irradiation in a controlled manner. That is, the processing apparatus 200 of the present embodiment can easily control the shape of the cell adhesion region 11a of the culture tool 100 by controlling the irradiation region. Also, the processing apparatus of the present embodiment can divide the irradiation region into segments based on the irradiation width of the laser L and irradiate the segments with the laser L. The irradiation width of the laser L can be adjusted to any desired width. For example, a narrower irradiation width of the laser allows more precise control of the shape, and a broader irradiation width of the laser allows a larger area to be irradiated with the laser L in a shorter time. Accordingly, the processing apparatus of the present embodiment is superior in terms of formability of the cell adhesion region 11a, for example.

Although the control unit 22 of the processing apparatus directly controls the laser irradiation unit 21 in the present embodiment, control of the laser irradiation unit 21 in the processing apparatus of the present invention is not limited thereto. As described above, when the processing apparatus includes a control unit for the laser irradiation unit 21 independently from the control unit 22, the position information acquisition section 225 writes, to this control unit for the laser irradiation unit 21, the segments L1 to L13 of the irradiation region and the coordinates of the corresponding endpoints and the laser ON/OFF switching positions associated with the segments L1 to L13. Then, based on the written information, the control unit for the laser irradiation unit 21 controls the laser irradiation unit 21 so as to apply the laser L to the photothermal conversion layer 13 of the culture tool 100.

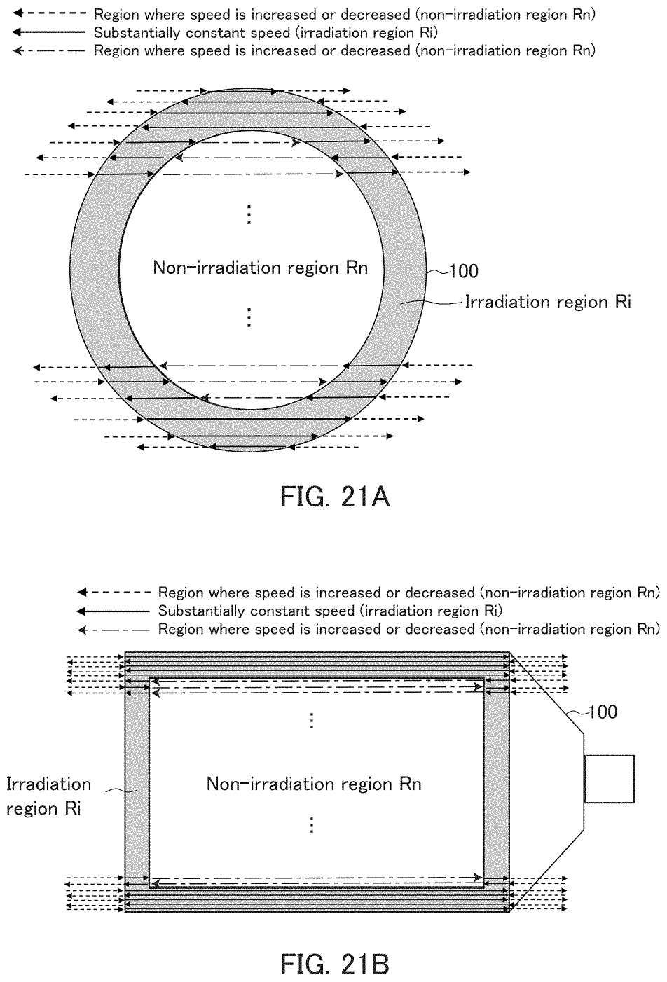

In the present embodiment, the irradiation position of the laser (a portion to be irradiated in the cell culture tool) is moved (scanned) at a substantially constant speed for both the irradiation region Rᵢ and the non-irradiation region Rₙ. However, the present invention is not limited thereto, and the irradiation control section 222 may change the moving speed of the irradiation position such that the moving speed differs between the irradiation region Rᵢ and the non-irradiation region Rₙ. Specifically, as shown in FIGS. 21A and 21B, the irradiation control section 22 may move the irradiation position of the laser at a substantially constant speed in the irradiation region Rᵢ, whereas the irradiation control section 22 may increase and/or decrease the moving speed of the irradiation position in the non-irradiation region Rₙ. In the case where the irradiation control section 222 changes the moving speed of the irradiated position of the laser such that it differs between the irradiation region Rᵢ and the non-irradiation region Rₙ, processing can be performed more quickly while maintaining the formability of the cell adhesion region 11a. Also, the irradiation control section 222 may increase and/or decrease the moving speed of the irradiation position of the laser in regions outside the cell culture tool 100.

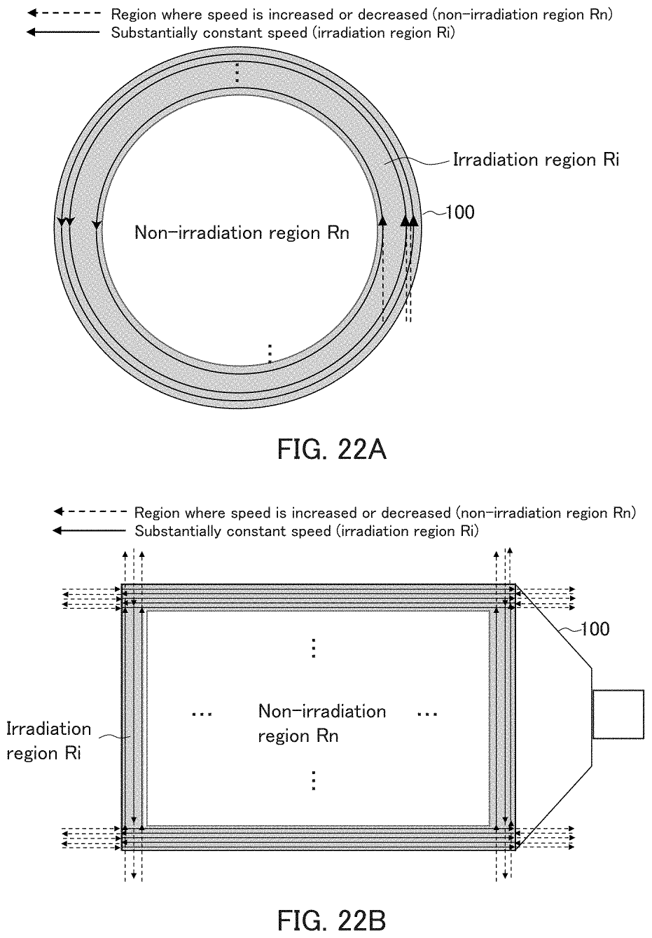

Although the dividing section 224 divides the irradiation region Rᵢ and the non-irradiation region Rₙ into strip-shaped segments in the present embodiment, the present invention is not limited thereto and the dividing section 224 may divide the irradiation region Rᵢ into segments with any desired shape. As a specific example, as shown in FIG. 22A, the dividing section 224 may divide the irradiation region Rᵢ into segments with an approximately circular (e.g., oval, circular, or perfectly circular) shape or with a spiral shape. By dividing the irradiation region Rᵢ in such a manner, the processing apparatus 200 can reduce the scanning distance of the laser and thus can perform processing more quickly. Although the dividing section 224 divides the irradiation region Rᵢ and the non-irradiation region Rₙ altogether in the present embodiment, the present invention is not limited thereto and only the irradiation region Rᵢ may be divided. As specific examples, as shown in FIGS. 22A and 22B, the dividing section 224 may divide only the irradiation region Rᵢ into strip-shaped segments or approximately circular segments. By dividing the irradiation region Rᵢ in such a manner, the processing apparatus 200 can perform processing more quickly, for example, in the case where an indistinct region is caused by the meniscus as described above.

### Fourth Embodiment

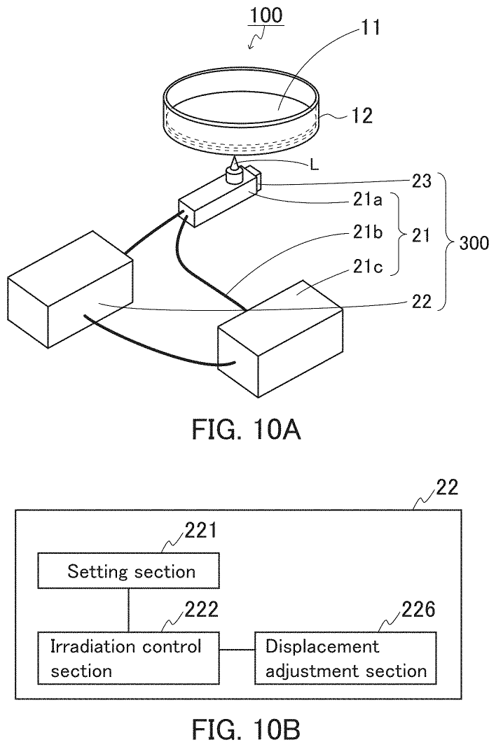

The present embodiment relates to still another example of the processing apparatus. FIG. 10 shows the structure of a processing apparatus 300 according to the present embodiment. FIG. 10 shows schematic views showing the structure of the processing apparatus 300 of the present embodiment. FIG. 10A is a perspective view showing an example of the structure of the processing apparatus 300 of the present embodiment. FIG. 10B is a block diagram showing an example of a control unit 22. As shown in FIG. 10A, the processing apparatus 300 of the present embodiment includes a displacement meter 23 as a displacement measurement section, in addition to the structural components of the processing apparatus 200 of the second embodiment. The displacement meter 23 is attached to a laser emission section 21a. Also, as shown in FIG. 10B, in the processing apparatus 300 of the present embodiment, the control unit 22 further includes a displacement adjustment section 226, in addition to the structural components of the control unit 22 of the processing apparatus 200 of the second embodiment. Except for the above, the structure of the processing apparatus 300 of the present embodiment is the same as the structure of the processing apparatus 200 of the second embodiment, and the above description regarding the processing apparatus 200 also applies to the processing apparatus 300.

The displacement meter 23 can measure the distance to the culture tool 100. The measurement system employed by the displacement meter 23 may be, for example, optical, eddy-current, ultrasonic, or laser force measurement system. When the displacement meter 23 and the displacement adjustment section 226 are provided as in the processing apparatus 300 of the present embodiment, the displacement adjustment section 226 moves the position of the laser emission section 21a based on the distance (displacement) measured by the displacement meter 23, whereby the laser L can be controlled so as to be focused on the photothermal conversion layer 13. With this structure, the processing apparatus 300 of the present embodiment can reduce strain and distortion of the culture tool 100 and thus can apply a desired light energy to the photothermal conversion layer 13.

The displacement meter 23 need only be capable of measuring the distance to the culture tool 100 as described above, and may be, for example, an optical observation device such as an optical microscope. In this case, the displacement meter 23 can measure the distance utilizing the focusing function on the bottom surface 12a of the culture tool 100. Specifically, the displacement meter 23 can back-calculate the distance from the optical observation device to the bottom surface 12a of the culture tool 100 based on the set value of the optical system when the optical observation device is focused on the bottom surface 12a. The displacement meter 23 measures the length in the height direction (the length in the direction orthogonal to the bottom surface 12a) to the culture tool 100, for example. In the present embodiment, the displacement meter 23 is attached to the laser emission section 21a and moves together with the laser emission section 21. However, the present invention is not limited thereto, and the displacement meter 23 may be arranged so as not to move together with the components of the laser irradiation unit 21, such as the laser emission section 21a. In this case, the displacement meter 23 is preferably arranged at a position where the displacement meter 23 does not moves together with the laser irradiation unit 21 in the height direction whereas it moves together with the laser irradiation unit 21 in a region where the position in the height direction does not change or in the X- and Y-axis directions. With this structure, the displacement meter 23 can measure the height to the culture tool 100 from a fixed position in the height direction regardless of the position of the laser irradiation unit 21.

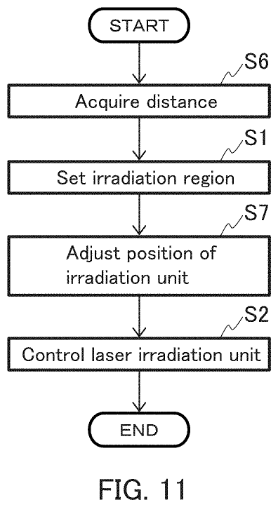

Next, a processing method using the processing apparatus 300 of the present embodiment will be described with reference to the flowchart of FIG. 11. FIG. 11 is a flowchart illustrating an example of processing (S1, S2, S6, and S7) performed by the processing apparatus 300.

First, in the step S6, the laser emission section 21a and the displacement meter 23 are placed below the bottom surface 12a of the culture tool 100. Preferably, they are placed below a central portion of the culture tool 100. Then, in the step S6, the distance, specifically the distance in the height direction, to the bottom surface 12a of the culture tool 100 is measured using the displacement meter 23. The displacement meter 23 is attached to the laser emission section 21a. Thus, in the step S6, taking the positional relationship between the laser emission section 21a and the displacement meter 23 into consideration, the height from the laser emission section 21a to the bottom surface 12a of the culture tool 100 is calculated based on the distance measured by the displacement meter 23.

Next, the step S1 is performed in the same manner as the step S1 in the second embodiment.

In the step S7, based on the distance acquired in the step S6, the displacement adjustment section 226 adjusts the position, specifically, the position in the height direction, of the laser irradiation unit 21. Specifically, the displacement adjustment section 226 adjusts the position of the laser irradiation unit 21 by controlling the above-described laser moving unit such that the laser L is focused on the photothermal conversion layer 13 when the laser irradiation unit 21 applies the laser L to the photothermal conversion layer 13. In the case where a reference value is set for the position of the laser irradiation unit 21 in the height direction, the displacement adjustment section 226 may adjust the position of the laser irradiation unit 21 in the height direction based on the reference value in the height direction and the distance acquired in the step S6.

Then, the step S2 is performed in the same manner as the step S2 in the second embodiment.

In the case where the photothermal conversion layer 13 converts the light energy of the laser L into thermal energy, it is preferable that the laser L is focused on the photothermal conversion layer 13. The position of the bottom surface 12a varies depending on the manufacturing lot of the culture tool 100, and the culture tool 100 may have a tilted or distorted bottom surface 12a. In this case, the position of the photothermal conversion layer 13 in the height direction is misaligned from the focal point of the laser L unless the height of the laser irradiation unit 21 is adjusted. As a result, the efficiency of converting the light energy of the laser L into thermal energy is reduced. According to the processing apparatus 300 of the present embodiment, the distance to the culture tool 100 is measured and the position of the laser irradiation unit 21 can be adjusted based on the thus-measured distance. This allows the laser L to be focused on the photothermal conversion layer 13, whereby the culture tool 100 can be processed efficiently.

### Fifth Embodiment

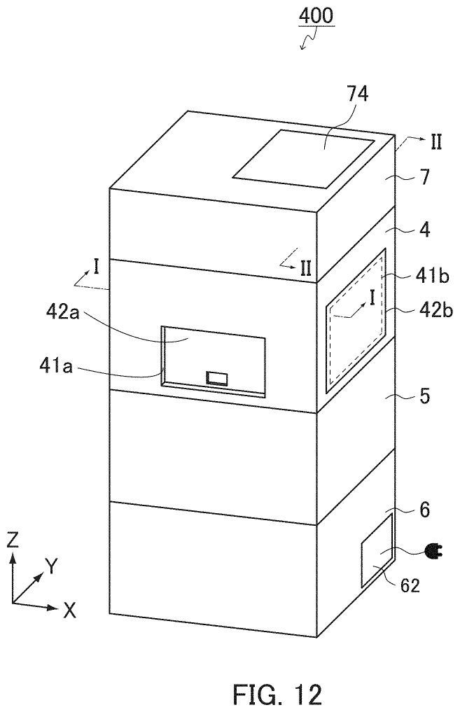

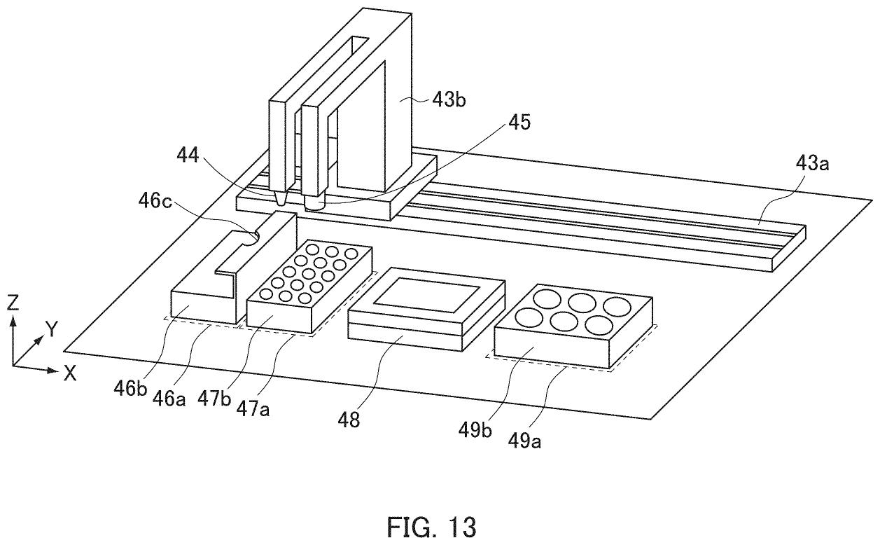

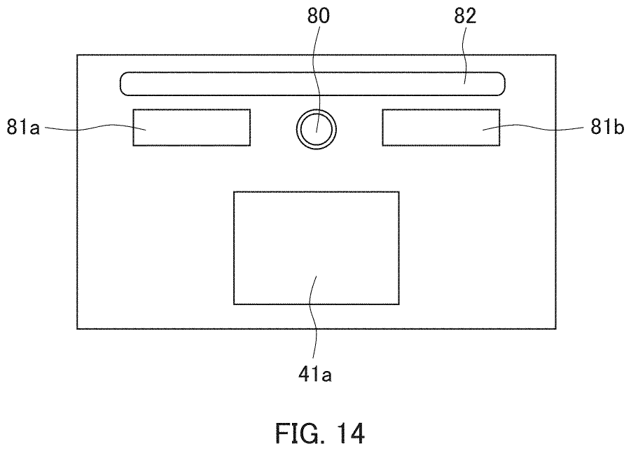

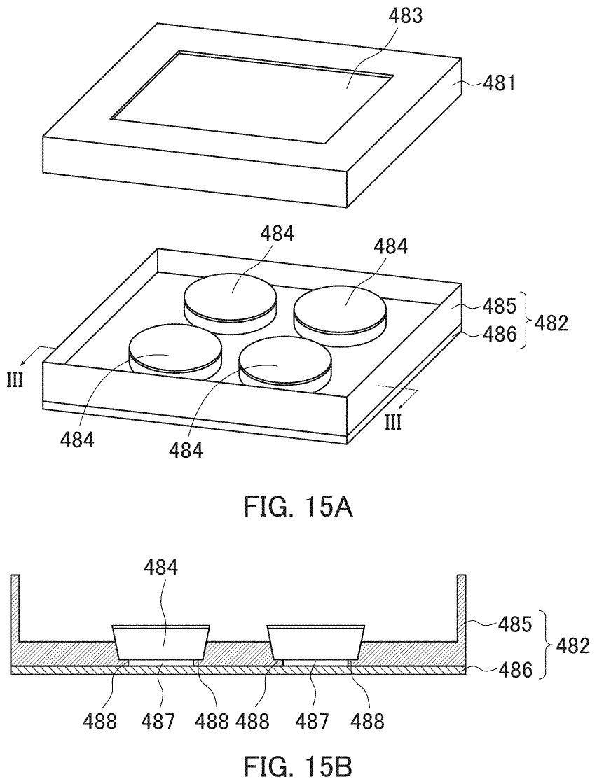

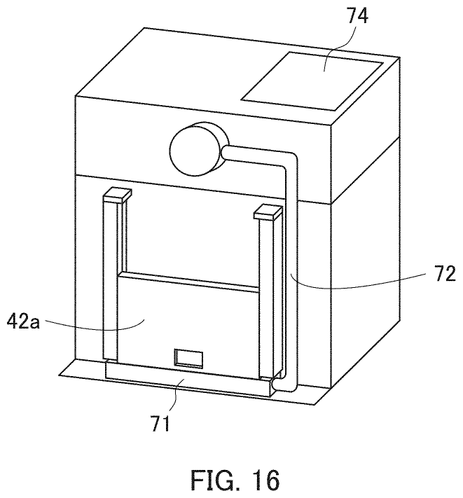

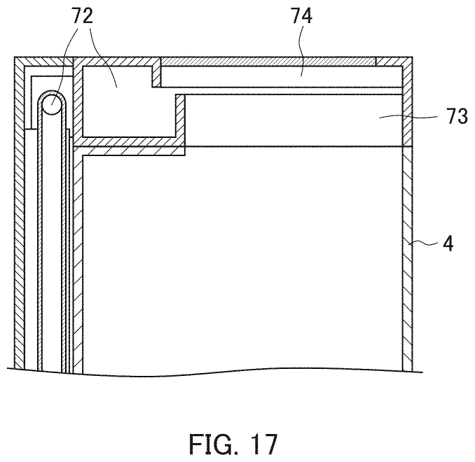

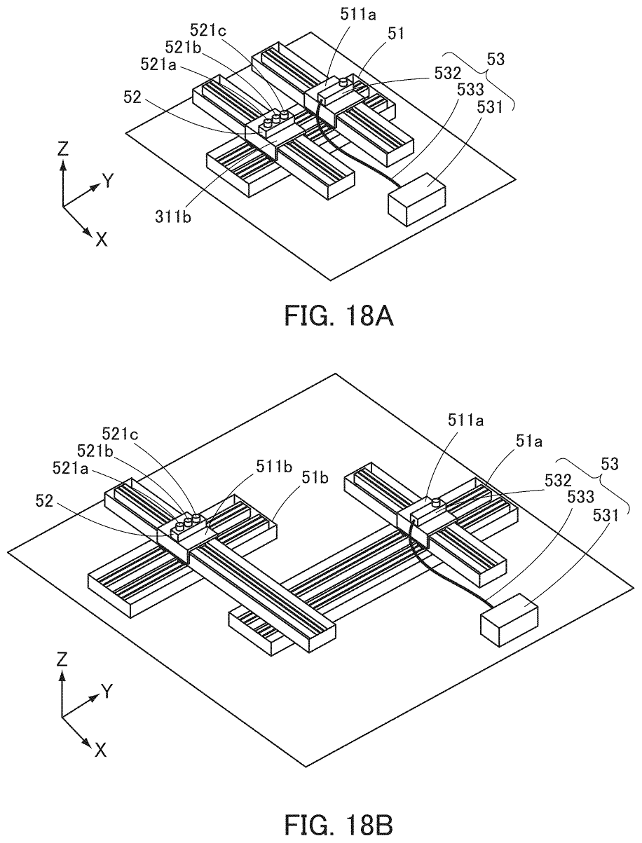

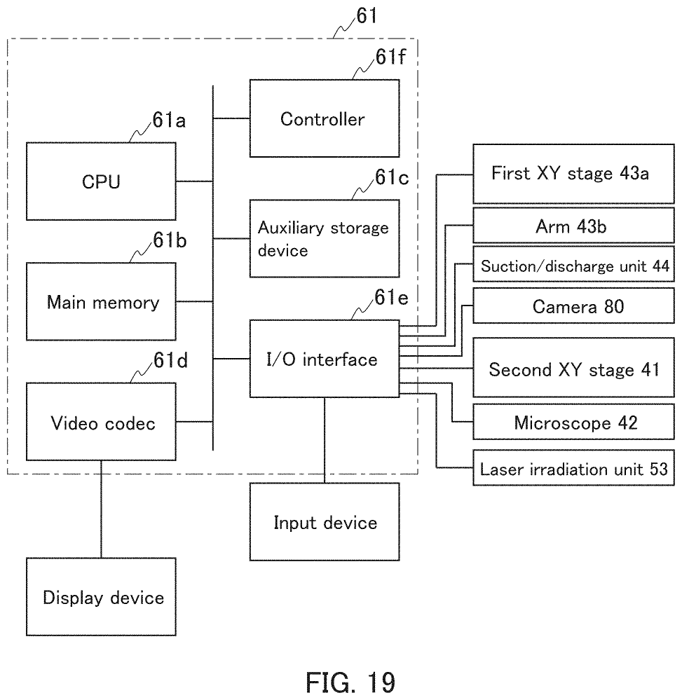

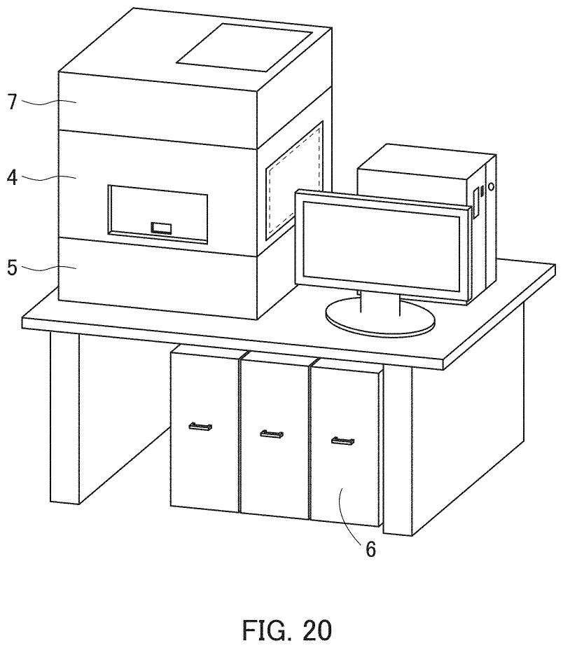

The present embodiment relates to still another example of the processing apparatus. FIGS. 12 to 20 show an example of the structure of the processing apparatus according to the present embodiment. FIG. 12 is a perspective view showing an example of the structure of the processing apparatus of the present embodiment. FIG. 13 is a perspective view showing an example of the structure of a first region in the processing apparatus of the present embodiment. FIG. 14 is a cross-sectional view of the first region as viewed along arrows I-I in FIG. 12. FIG. 15A is an exploded perspective view showing an example of a tool placement portion in the processing apparatus of the present embodiment and FIG. 15B is a cross-sectional view as viewed along arrows III-III in FIG. 15A. FIG. 16 is a perspective view of the first region and a circulation unit in a state where an outer wall of the first region is removed. FIG. 17 is a cross-sectional view of an upper part of the first region and the circulation unit as viewed along arrows II-II in FIG. 12. FIG. 18A is a perspective view showing an example of the structure of a second region in the processing apparatus of the present embodiment and FIG. 18B is a perspective view showing another example of the structure of the second region. FIG. 19 is a block diagram showing an example of a control section of the processing apparatus of the present embodiment. FIG. 20 is a perspective view showing another example of the structure of the processing apparatus of the present embodiment.

As shown in FIG. 12, a processing apparatus 400 of the present embodiment includes a first region 4, a second region 5, a third region 6, and a circulation unit 7, and the first region 4, the second region 3, and the third region 6 are arranged in succession in this order from the top toward the bottom. Although the processing apparatus 400 of the present embodiment includes the circulation unit 7, the circulation unit 7 is an optional component and may or may not be present. The positional relationship among the first region 4, the second region 5, and the third region 6 need only be such that the first region 4 and the second region 5 are arranged in succession (adjacent to each other), and the third region 6 may be arranged at any position. For example, as shown in FIG. 20, the third region 6 may be arranged spaced apart from the first region 4 and the second region 5. In the case where the third region 6 is arranged spaced apart from the first region 4 and the second region 5 as shown in FIG. 20, the processing apparatus 400 can also be referred to as a processing system, for example. The processing system may be a tabletop system, for example. The first region 4 is preferably arranged on the top of the second region 5. When laser irradiation is performed from above a culture tool 100 using a laser irradiation unit 53 to be described below, it is necessary to place an emission aperture of a laser emission section 532 in a solution contained in the culture tool 100 in order to stabilize the focal point of the laser irradiation unit 53. However, performing laser irradiation in this state causes a problem such as sticking or baking of components of the solution to the emission aperture of the laser emission section 532, resulting in contamination of the emission aperture of the laser emission section 532. Thus, by arranging the laser irradiation unit 53 as in the processing apparatus 400 of the present embodiment, it is possible to suppress the occurrence of contamination of the laser emission aperture of the laser irradiation unit 53 at the time of irradiating the photothermal conversion layer 13 in the culture tool 100 with the laser L using the laser irradiation unit 53 to be described below, for example. Accordingly, the processing apparatus 400 of the present invention can stabilize the output power of the laser emitted from the laser irradiation unit 53 and thus can efficiently process the culture tool 100, for example. The material for forming each region is not limited to particular materials, and each region may be formed of, for example, a stainless steel plate, a rust-proof iron plate, and a resin plate that can be molded by vacuum molding, injection molding, compressed-air molding, or the like. The material for forming each region is preferably a non-light transmitting material because this allow a second imaging unit to be described below to capture clearer images of the inside of the culture tool 100. The term “non-light transmitting” means that, for example, transmission of light having a wavelength that affects image capturing by the second imaging unit is suppressed. When the second imaging unit is a fluorescence microscope, the wavelength of the above-described light may be a wavelength corresponding to fluorescence to be detected, for example. Specific examples of the non-light transmitting material include the above-described materials for forming each region. The size and shape of each region are not limited to particular sizes and shapes, and can be set as appropriate according to the size and shape of each member (unit) to be placed in each region. In the processing apparatus 400 of the present embodiment, the first region 4 and the second region 5 are constituted by different housings, and the housings constituting the first region 4 and the second region 5 are arranged adjacent to each other. However, the present invention is not limited thereto, and the first region 4 and the second region 5 may be constituted by a single housing with the inner space of this housing being partitioned to provide the first region 4 and the second region 5. By using different housings to constitute the first region 1 and the second region 5 as in the processing apparatus 400 of the present embodiment, maintenance of each member of the processing apparatus 400 can be performed easily and the processing apparatus 400 can be assembled easily, for example.

The first region 4 includes an opening 41a for operations on its front surface (on the front side in FIG. 12) and an opening 41b for enabling maintenance on its side surface. The opening 41a is an opening for operations relating to processing of the culture tool 100 in a processing chamber in the first region 4. The opening 41b is an opening for enabling maintenance of the processing chamber. The opening 41a preferably has a smaller opening area than the opening 41b because this allows the maintenance operations to be performed more easily, for example. There is no particular limitation on the size and the number of openings 41a and 41b, and reference can be made to, for example, the size and the number of openings for operations and openings for enabling maintenance in safety cabinets. As a specific example, for the size and the number of openings 41a and 41b, reference can be made to, for example, the standards for safety cabinets as specified in EN standards, namely, EN 12469:2000. The number of openings 41b is not limited and can be set freely. It is preferable to provide two or more openings 41b because, for example, maintenance can be performed more easily. The positions at which the openings 41a and 41b are arranged in the first region 4 are not limited to particular positions and can be set freely. Preferably, the openings 41a and 41b are arranged at different positions (e.g., on different side surfaces) in the first region 4. Although the opening 41b is primarily intended to facilitate the maintenance in the processing apparatus 400 in the present embodiment, the opening 41b may also be used for other purposes. In the processing apparatus 400 of the present embodiment, for example, movement and the like of each member arranged inside can be observed through the opening 41b. This allows a defective area to be observed directly when a trouble occurs in the processing apparatus 400, and a user thus can consider countermeasures.

The front wall of the first region 4 is a double wall having an outer wall and an inner wall, and the opening 41a is opened/closed by moving a door 42a up/down along rails provided in a space between the outer wall and the inner wall. The opening 41b can be opened/closed by detaching/attaching a door 42b that covers the opening. The opening 41b is preferably sealed with the door 42b when processing the culture tool 100 in the processing chamber, for example. This can prevent, for example, the gas outside the processing apparatus 400 and the dust contained therein from flowing into the processing chamber. In the processing apparatus 400 of the present embodiment, the opening 41a and the door 42a thereof and the opening 41b and the door 42b thereof are optional components, and the processing apparatus 400 may or may not include them or may include either one of the openings and the door thereof. The wall of the first region 4 may be either a double wall or a single wall, and preferably is the former because the size of the processing apparatus 400 can be reduced by arranging other members inside the double wall. When the wall of the first region 1 is a single wall, the door 42a is arranged, for example, outside the first region 4, as with the door 42b. The opening and closing system of each door is not limited to particular systems. For example, the door may be a liftable door like the door 42a, an externally attached door like the door 42b, or any other type of door. The other type of door is, for example, a hinged double door, accordion door, or sliding door. The material for forming each door is not limited to particular materials. Examples thereof include the above-described materials for forming each region, and non-light transmitting materials are preferable.

As shown in FIG. 13, the inner space of the first region 4 of the processing apparatus 400 of the present embodiment is the processing chamber in which the culture tool 100 is processed. The processing chamber can be closed by closing the doors 42a and 42b. In other words, the processing chamber is openable and closable. The processing chamber includes: an XY stage 43a and an arm 43b that collectively constitutes a suction/discharge moving unit; a suction/discharge unit 44; a light source 45; a drainage container placement portion 46a; a storage container placement portion 47a; a tool placement portion 48; and a collection container placement portion 49a. Although the processing chamber includes the XY stage 43a, the arm 43b, the suction/discharge unit 44, the light source 45, the drainage container placement portion 46a, the storage container placement portion 47a, and the collection container placement portion 49a in the present embodiment, they are all optional components and may or may not be present. Alternatively, the processing chamber may include any one of them or two or more of them. The XY stage 43a is arranged on the bottom surface of the processing chamber so as to be movable in the directions of arrows X and Y The arm 43b including a pair of arms is arranged on the top of the XY stage 43a. At the tip of one of the arms in the arm 43b, the suction/discharge unit 44 is arranged with its suction/discharge port directed downward. At the tip of the other arm in the arm 43b, the light source 45 is arranged so as to be capable of projecting (emitting) light downward. The drainage container placement portion 46a, the storage container placement portion 47a, the tool placement portion 48, and the collection container placement portion 49a are arranged on the bottom surface of the processing chamber in this order along the moving direction of the XY stage 43a, which is the direction of arrow X. A drainage container 46b including a tip member detachment unit 46c is placed in the drainage container placement portion 46a, a storage container 47b is placed in the storage container placement portion 47a, and a collection container 49b is placed in the collection container placement portion 49a.

Although the XY stage 43a and the arm 43b are provided as the suction/discharge moving unit in the processing apparatus 400 of the present embodiment, the suction/discharge moving unit is not limited thereto. The suction/discharge moving unit need only be capable of moving the suction/discharge unit 44, and a known moving unit can be used, for example. The moving direction of the suction/discharge moving unit is not limited to particular directions, and the suction/discharge moving unit may be, for example, movable in one direction (e.g., the direction of arrow Y), movable in two directions (e.g., the directions of arrows X and Y), or movable in three directions (e.g., the directions of arrows X, Y, and Z). In the case of two moving directions, the first direction need only be nonparallel to the second direction and is preferably orthogonal or substantially orthogonal to the second direction. In this case, a plane including the first direction and the second direction is preferably substantially parallel to the surface on which the tool placement portion 48 is arranged. In the case of three moving directions, the third direction need only intersect with a plane including the first direction and the second direction, for example, and is preferably orthogonal or substantially orthogonal to the plane including the first direction and the second direction. In the present embodiment, the XY stage 43a is a known stage capable of moving an object at high speed and precisely along the directions of arrows X and Y via, for example, a linear motor carriage. The arm 43b is extendable in the vertical direction (the direction of arrow Z). The arm 43b, however, may be fixed. In the latter case, the suction/discharge moving unit is capable of moving the suction/discharge unit 44 only in a plane substantially parallel to the bottom surface of the processing chamber, i.e., only in the directions of arrows X and Y in FIG. 13.

The suction/discharge unit 44 sucks and discharges a solution, such as a culture medium, and cells in the culture tool 100, for example. The suction/discharge unit 44 is used, for example, with a tip member to be described below attached to the suction/discharge port side thereof. The suction/discharge unit 44 is not limited to particular types of units, and a known suction/discharge unit can be used, for example. Specific examples thereof include electric pipette and electric syringe pumps.

The light source 45 emits light toward the tool placement portion 48 from above the tool placement portion 48, for example. For example, when an optical microscope such as a phase-contrast microscope is used as a second imaging unit to be described below, it is preferable to use the light source 45 in combination. Light emitted by the light source 45 is visible light, for example. The light source 45 is not limited to particular types of light sources, and may be, for example, a known light source such as a xenon light source, a light emitting diode (LED) lighting device, or a laser diode (LD). In the present embodiment, the light source 45 is arranged in the arm 43b of the suction/discharge moving unit and moves synchronously with the suction/discharge unit 44. However, the light source 45 may move asynchronously with the suction/discharge unit 44. As a specific example, the light source 45 may be arranged in, for example, a light source moving unit that is different from the suction/discharge moving unit and capable of moving the light source 45. In this case, a control unit 61 to be described below may include a light source movement control unit that controls the movement of the light source moving unit. For example, the above description regarding the moving direction of the suction/discharge unit also applies to the moving direction of the light source moving unit.

The drainage container placement portion 46a is a portion in which a drainage container 46b for draining a liquid sucked by the suction/discharge unit 44 can be placed. Although the drainage container 46b is placed in the drainage container placement portion 46a in the present embodiment, the drainage container 46b is an optional component and may or may not be present. In the present embodiment, the drainage container 46b is an open-topped box that has a wall extending upward on the storage container placement portion 47a side, and at the upper end of this wall, a wall (upper surface) including a tip member detachment unit 46c formed as a semicircular recess (notch) extends substantially parallel to the bottom surface of the processing chamber. Since the drainage container 46b can collect a tip member detached from the suction/discharge unit 44, the drainage container 46b can also be referred to as, for example, a tip member collection container, and the drainage container placement portion 46a can also be referred to as a tip member collection container placement portion. Although the tip member detachment unit 46c is formed in the drainage container 46b, it may be provided separately from the drainage container 46b. The tip member detachment unit 46c may be arranged near the suction/discharge unit 44, specifically, in the suction/discharge moving unit in which the suction/discharge unit 44 is arranged.

The storage container placement portion 47a is a portion in which the storage container 47b containing the tip member detachable from the suction/discharge unit 44 can be placed. Although the storage container 47b is placed in the storage container placement portion 47a in the present embodiment, the storage container 47b is an optional component and may or may not be present. The tip member is not limited to particular members and need only be capable of storing a liquid sucked by the suction/discharge unit 44 therein. For example, when the suction/discharge unit 44 is a pipette, the tip member may be a tip. The storage container 47b is, for example, a rack in which the tips are stored. The processing apparatus 400 of the present embodiment includes the tip member detachment unit 46c and the storage container placement portion 47a, and with this structure, the movement at the time of sucking and discharging a solution, such as a culture medium, cells, and the like in the culture tool 100 can be simplified (shortened).

The collection container placement portion 49a is a portion in which the collection container 49b for collecting the sucked liquid containing cells collected by the suction/discharge unit 44 can be placed. Although the collection container 49b is placed in the collection container placement portion 49a in the present embodiment, the collection container 49b is an optional component and may or may not be present. The collection container 49b may be, for example, a culture vessel such as a known dish or a known flask.

In the present embodiment, the drainage container placement portion 46a, the storage container placement portion 47a, the tool placement portion 48, and the collection container placement portion 49a are arranged in this order on the bottom surface of the processing chamber along the moving direction of the XY stage 43a, which is the long axis direction (the direction of arrow X) in a plane substantially parallel to the surface on which the tool placement portion 48 is arranged, i.e., the bottom surface of the processing chamber. However, the respective placement portions need not be arranged along the long axis direction and need not be arranged in this order. In the present embodiment, the drainage container placement portion 46a, the storage container placement portion 47a, the tool placement portion 48, and the collection container placement portion 49a are arranged in the above-described order. With this structure, for example, the suction/discharge unit 44 can move linearly, and the movement at the time of sucking and discharging a solution, such as a culture medium, cells, and the like in the culture tool 100 can be simplified (shortened).

As shown in FIG. 14, a first camera 80, illumination lamps 81a and 81b, and a germicidal lamp 82 are arranged above the opening 41a on the front wall of the processing chamber of the processing apparatus 400 of the present embodiment. The illumination lamps 81a and 81b are arranged on both sides of the first camera 80 in the direction of arrow X, and the germicidal lamp 82 is arranged above the first camera 80.

Although the camera 80 is provided as a first imaging unit in the present embodiment, the first imaging unit is an optional component and may or may not be present. The first imaging unit is not limited to a camera and need only be capable of capturing images of the inside of the processing chamber. The first imaging unit is not limited to particular types of imaging units, and may be a known imaging unit such as a microscope or a camera, which may be used in combination with a solid-state imaging element (image sensor) such as a CCD or a complementary MOS (CMOS). Although the camera 80 is arranged on the front wall inside the processing chamber in the present embodiment, the position of the camera 80 is not limited to particular positions and can be set freely. The camera 80 is preferably arranged such that it can capture images of a wide range of area in the processing chamber. Specifically, in the case where the XY stage 43a and the arm 43b, which collectively constitute the suction/discharge moving unit, and the suction/discharge unit 44 are arranged on the back side (the upper left side in FIG. 13) of the tool placement portion 48 in the processing chamber as in the processing apparatus 400 of the present embodiment, it is preferable to arrange the camera 80 on the front side (the lower right side in FIG. 13) of the processing chamber because this allows the camera 80 to capture images of a wide range of area in the processing chamber. The first imaging unit is preferably capable of capturing images at a plurality of magnifications (e.g., different magnifications), but may be capable of capturing images at one magnification. The magnification means an imaging magnification, for example. As a specific example, the camera 80 includes, for example, lenses with a plurality of magnifications (e.g., different magnifications). The first imaging unit may be capable of, for example, optical zooming or digital zooming. The processing apparatus 400 of the present embodiment includes the camera 80, and this allows, for example, operations in the processing chamber to be checked, whereby the reliability of the operations is improved. The number of first imaging units arranged in the processing chamber is not limited, and may be one or more.

Although the illumination lamps 81a and 81b are provided as illumination units in the present embodiment, the illumination units are optional components and may or may not be present. The illumination unit is not limited to an illumination lamp and need only be capable of projecting light to (illuminating) the processing chamber. The illumination unit is not limited to particular types of illumination units, and for example, a known illuminating device such as a fluorescent lamp or a LED lamp can be used. Although the illumination lamps 81a and 81b are arranged on the front wall inside the processing chamber in the present embodiment, the positions of the illumination lamps 81a and 81b are not limited to particular positions and can be set freely. The illumination lamps 81a and 81b are preferably arranged such that they can project light to a wide range of area in the processing chamber, i.e., formation of shadows in the processing chamber is avoided as much as possible. Specifically, in the case where the XY stage 43a and the arm 43b, which collectively constitute the suction/discharge moving unit, and the suction/discharge unit 44 are arranged on the back side (the upper left side in FIG. 13) of the tool placement portion 48 in the processing chamber as in the processing apparatus 400 of the present embodiment, it is preferable to arrange the illumination lamps 81a and 81b on the front side (the lower right side in FIG. 13) of the processing chamber because this allows the illumination lamps 81a and 81b to project light to a wide range of area in the processing chamber. The processing apparatus 400 of the present embodiment includes the illumination lamps 81a and 81b, and this allows, for example, operations in the processing chamber to be checked, whereby the reliability of the operations is improved. The number of the illumination units arranged in the processing chamber is not limited, and may be one or more.

Although the germicidal lamp 82 is provided as a germicidal unit in the present embodiment, the germicidal unit is an optional component and may or may not be present. The germicidal unit is not limited to a germicidal lamp and need only be capable of disinfecting the inside of the processing chamber, especially a portion around the tool placement portion 48. The germicidal unit is not limited to particular types of germicidal units, and for example, a known germicidal unit such as a germicidal lamp or an ultraviolet LED lamp can be used. Although the germicidal lamp 82 is arranged on the front wall inside the processing chamber in the present embodiment, the position of the germicidal lamp 82 is not limited to particular positions and can be set freely. For example, dust and the like outside the processing apparatus 400 flows in via the openings 41a and 41b. Thus, the germicidal lamp 82 is preferably arranged such that it can disinfect areas near the openings 41a and 41b. Specifically, in the case where the front wall of the processing chamber has the opening 41a as in the processing apparatus 400 of the present embodiment, the germicidal unit is preferably arranged above the opening 41a on the front wall of the processing chamber. Also, in the case where the side wall of the processing chamber has the opening 41b as in the processing apparatus 400 of the present embodiment, the germicidal unit is preferably arranged above the opening 41b on the side wall of the processing chamber. Moreover, in the case where the processing apparatus 400 includes the illumination unit and the germicidal unit, both of these units are preferably arranged on the same wall of the processing chamber, e.g., on the wall with the opening 41a. In this case, it is preferable to arrange the germicidal unit above the illumination unit. The processing apparatus 400 of the present embodiment includes the germicidal lamp 82, and this improves the cleanliness inside the processing chamber, for example. The number of germicidal units arranged in the processing chamber is not limited, and may be one or more.

For the size, shape, structure, and the like of the processing chamber in the first region 4 of the present embodiment, reference can be made to, for example, the size, shape, structure, and the like of safety cabinets, and as a specific example, reference can be made to the standards for safety cabinets as specified in EN 12469:2000 as described above.

As shown in FIG. 15, the tool placement portion 48 in the processing apparatus 400 of the present embodiment includes an upper lid 481 and a bottom portion 482, and the upper lid 481 is detachably attached to the bottom portion 482. In the present embodiment, the tool placement portion 48 is a box including the upper lid 481 and the bottom portion 482, and the culture tool 100 is placed inside the box. However, the tool placement portion 48 is not limited thereto, and need only be configured such that: the culture tool 100 can be placed therein; the tool placement portion 48 is arranged in the processing chamber so as to be adjacent to the second region 5; and the adjacent portion (a bottom plate 486 in FIG. 15) of the tool placement portion 48 to the second region 5 is capable of transmitting light. “Transmitting light” described above means, for example, allowing a laser emitted from by the laser irradiation unit 53 arranged in the second region 5 to pass therethrough. In the case where the second region 5 includes a second imaging unit to be described below, “transmitting light” means that the second imaging unit can capture images through the bottom plate 486. The upper lid 481 has a light transmitting region 483 to allow the culture tool 100 to be irradiated with light from the light source 45. The light transmitting region 483 is formed of a transparent glass plate or a transparent acrylic plate, for example. The bottom portion 482 includes a bottom wall 485 and the light-transmitting bottom plate 486. The light-transmitting bottom plate 486 is formed of a transparent glass plate or a transparent acrylic plate, for example. The bottom plate 486 is adjacent to the second region 5. Thus, it can also be said that the adjacent portion of the tool placement portion 48 to the second region 5, i.e., the bottom plate 486, forms a part of the wall of the processing chamber. The contact portion between the bottom plate 486 and the wall of the processing chamber is preferably sealed with a sealing member such as a gasket or a sealing material, for example. This can prevent, for example, the gas in the second region 5 and dust and the like contained therein from flowing into the tool placement portion 48 and the processing chamber. The bottom wall 485 includes four recesses 487 in which four culture tools 100 can be placed, respectively, and the side surface of each recess 487 has an inversely tapered shape that narrows from the inside toward the outside of the processing chamber (from the top toward the bottom in FIG. 15B). Each recess 487 includes a protruding portion 488 protruding inward at its end on the bottom plate 486 side. The bottom end of the culture tool 100 comes in contact with the protruding portion 488. Although the bottom wall 485 has four recesses 487 in the processing apparatus 400 of the present embodiment, the number of recesses 487 provided in the bottom wall 485 is not limited thereto, and can be set as appropriate according to the number of culture tools 100 to be placed. The size of each recess 487 can be set as appropriate according to the size of the culture tool 100 to be placed therein. With the above-described structure of the recess 487 in the tool placement portion 48 of the present embodiment, it becomes possible to place the culture tool 100 in the tool placement portion 48 regardless of the shape of the side surface of the culture tool 100, for example. In the processing apparatus 400 of the present embodiment, the bottom wall 485 is formed in one piece by integrally forming its bottom surface wall and side walls. However, the bottom wall 485 is not limited thereto, and its bottom surface wall and side walls may be provided as separate members. When the bottom wall 485 is constituted by separate members, two or more types of members that differ from each other in, for example, the number and the size of recesses 487 provided therein can be prepared as members constituting the bottom surface wall of the bottom wall 485. Thus, for example, according to the size and the number of culture tools 100, the bottom surface wall of the bottom wall 485 can be replaced with the member with the size and the number of recesses suitable for the placement of the culture tools 100, thereby allowing the culture tools 100 to be placed suitably.

The tool placement portion 48 may further include, for example, a temperature adjustment unit for adjusting the temperature of the culture tool 100. The temperature adjustment unit allows culture conditions during culture of cells in the culture tool 100 to be kept constant, whereby damage to the cells during the cell culture can be reduced, for example. The temperature adjustment unit may be, for example, a heating unit such as a heater.

The tool placement portion 48 may further include, for example, a pH adjustment unit for adjusting the pH of a solution, such as a culture medium, in the culture tool 100. When the tool placement portion 48 includes the pH adjustment unit, culture conditions during culture of cells in the culture tool 100 can be kept constant, whereby damage to the cells during the cell culture can be reduced, for example. The pH adjustment unit may be, for example, a carbon dioxide concentration adjustment unit, which specifically may be, for example, a carbon dioxide cylinder or a connector for connection with a carbon dioxide supply unit provided outside the processing apparatus 400.

As shown in FIGS. 16 and 17, in the processing apparatus 400 of the present embodiment, the circulation unit 7 includes an intake section 71, a circulation path 72, a gas supply section 73, and an exhaust section 74. With this structure, the circulation unit 7 circulates the gas inside the processing chamber.

The intake section 71 sucks the gas in the processing chamber. The intake section 71 may suck gas outside the processing apparatus 400 instead of or in addition to the gas inside the processing chamber. In the present embodiment, the intake section 71 is arranged near (e.g., immediately below) the opening 41a of the processing chamber. Specifically, the intake section 71 has a plurality of openings (not shown, e.g., slits) formed on its upper surface and is arranged below the opening 41a such that these openings are in communication with the opening 41a. By arranging the intake section 71 near the opening 41a of the processing chamber as described above, it becomes possible to prevent, for example, gas outside the processing apparatus 400 and dust and the like contained therein from flowing into the processing chamber when an operator opens the door 42a and performs operations in the processing chamber. The intake section 71 may be arranged at a position near the opening 41b, instead of or in addition to a position near the opening 41a. The intake section 71 may suck the gas inside the processing chamber using an air blowing unit such as a fan, for example.

The circulation path 72 connects the intake section 71 to the gas supply section 73 and the exhaust section 74. In the present embodiment, the circulation path 72 is arranged in a space between the outer wall and the inner wall and on the top of the first region 4. The circulation path 72 is a hollow tube, for example. One end of the circulation path 72 is in communication with the intake section 71, and the other end of the circulation path 72 is in communication with the gas supply section 73 and the exhaust section 74. By arranging the circulation path 72 in the space between the outer wall and the inner wall as in the processing apparatus 400 of the present embodiment, the size of the processing apparatus 400 can be reduced, for example. Although the circulation unit 7 includes the circulation path 72 in the present embodiment, the circulation path 72 may or may not be present. In the latter case, the intake section 71 is directly connected to the gas supply section 73 and the exhaust section 74, for example. The circulation path 72 may feed the gas sucked by the intake section 71 to the gas supply section 73 and the exhaust section 74 using the air blowing unit such as a fan, for example.

When the circulation path 72 includes the air blowing unit, the air blowing unit may be arranged near the intake section 71, the gas supply section 73, or the exhaust section 74, or may be arranged at any other position such as a central portion of any of these sections. However, it is preferable to arrange the air blowing unit near the intake section 71 because this improves suction by the intake section 71, whereby the dust and the like can be more effectively prevented from flowing into the processing chamber, for example, as compared with the downflow caused by the gas supply section 73 to be described below. In the case where air blowing unit is arranged near the intake section 71, the air blowing unit is preferably arranged in, for example, the second region 5 or the third region 6. As a specific example, when the circulation path 72 further includes the air blowing unit in the processing apparatus 400 of the present embodiment, the air blowing unit is arranged on the front side (the lower left side in FIG. 12), i.e., below the intake section 71, in the second region 5 or the third region 6. In this case, the circulation path 72 connects the intake section 71 to the intake side of the air blowing unit and also connects the blowing side of the air blowing unit to the gas supply section 73 and the exhaust section 74. That is, the circulation path 72 is arranged so as to extend in the second region 5 or in the second region 5 and the third region 6, in the space between the outer wall and the inner wall, and above the first region 4.

The gas supply section 73 supplies part of the gas sucked by the intake section 71 into the processing chamber. In the present embodiment, the gas supply section 73 is in communication with the upper end of the first region 4 such that the gas sucked by the intake section 71 can be supplied into the processing chamber. The gas supply section 73 may supply gas into the processing chamber using the air blowing unit such as a fan, for example. The gas supply section 73 may also include, for example, a gas purification unit. In this case, gas supplied from the gas supply section 73 into the processing chamber passes through the gas purification unit. When the gas supply unit 73 includes the gas purification unit, it is possible to prevent the dust and the like from flowing into the processing chamber, for example. The gas purification unit may be, for example, a filter for collecting fine particulates, such as a high efficiency particulate air filter (HEPA filter) or an ultra-low penetration air filter (ULPA filter). In the processing apparatus 400 of the present embodiment, the upper part of the processing chamber is connected to the gas supply section 73. With this structure, for example, downflow is caused by the gas blown from the gas supply section 73, whereby dust and the like can be more effectively prevented from flowing into the processing chamber from the opening 41a.

The exhaust section 74 discharges the remainder of the gas sucked by the intake section 71 to the outside of the processing chamber, specifically, to the outside of the processing apparatus 400. In the present embodiment, the exhaust section 74 is placed at an upper end (the uppermost part) of the processing apparatus 400 such that the gas sucked by the intake section 71 can be discharged to the outside of the processing apparatus 400. When the exhaust section 74 is provided in the uppermost part of the processing apparatus 400 as described above, the size of the processing apparatus 400 can be reduced and dust stirred up by the discharged gas can be prevented from flowing into the processing chamber, for example. The exhaust section 74 may discharge gas to the outside of the processing apparatus 400 using the air blowing unit such as a fan, for example. The exhaust section 74 may also include the gas purification unit, for example. In this case, gas discharged to the outside of the processing apparatus 400 from the exhaust section 74 passes through the gas purification unit. When the exhaust section 74 includes the gas purification unit, fine particles and the like generated in the processing chamber can be prevented from flowing out to the outside of the processing apparatus 400, for example.

For the size, shape, structure, and the like of each component of the circulation unit 7 of the present embodiment, reference can be made to, for example, the size, shape, structure, and the like of safety cabinets, and as a specific example, reference can be made to the standards for safety cabinets as specified in EN 12469:2000 as described above.

As shown in FIG. 18A, in the processing apparatus 400 of the present embodiment, the second region 5 includes a second XY stage 51, a microscope 52 having objective lenses 521a to 521c with three different magnifications, and a laser irradiation unit 53. Although the processing apparatus 400 of the present embodiment includes the XY stage 51 and the microscope 52, the XY stage 51 and the microscope 52 are optional components and the processing apparatus 400 may or may not include them or may include either one of them. The XY stage 51 is arranged on the bottom surface of the second region 5, which is substantially parallel to the surface on which the tool placement portion 48 is arranged, i.e., the bottom surface of the processing chamber. In the XY stage 51, two rails extending in the direction of arrow X are arranged on a common rail (moving path) extending in the direction of arrow Y so as to be movable on the common rail. Carriages 511a and 511b are arranged on the two rails extending in the direction of arrow X, respectively, so as to be movable on the rails. The laser irradiation unit 53 includes a laser source 531, a laser emission section 532, and an optical fiber 533. On the top of the XY stage 51, the microscope 52 is arranged on the carriage 511b with the objective lenses 521a to 521c facing upward (the direction of arrow Z), and the laser emission section 532 of the laser irradiation unit 53 is arranged on the carriage 511a with a laser emission aperture facing upward (the direction of arrow Z). The carriage 511a is movable up and down in the vertical direction (the direction of arrow Z). In the second region 5, the laser source 531 is arranged on the bottom surface of a portion of the second region 5 that does not overlap the movable range of the XY stage 51. One end of the optical fiber 533 is connected to the laser source 531, and the other end is connected to the laser emission section 532.

Although the XY stage 51 is provided as a laser moving unit and a second imaging moving unit in the processing apparatus 400 of the present embodiment, the laser moving unit and the second imaging moving unit are not limited thereto. The laser moving unit and the second imaging moving unit need only be capable of moving the laser irradiation unit 53 and a second imaging unit to be described below, respectively, and known moving units can be used, for example. In the present embodiment, the laser moving unit and the second imaging moving unit share the rail extending in the direction of arrow X (first direction). However, the laser moving unit and the second imaging moving unit may be independent from each other. As a specific example, as shown in FIG. 18B, the laser moving unit may be arranged as, for example, an XY stage 51a and the second imaging moving unit may be arranged as an XY stage 51b on the bottom surface of the second region 5. The moving directions of the laser moving unit and the second imaging moving unit are not limited to particular directions, and these units may be, for example, movable in one direction (e.g., the direction of arrow Y), movable in two directions (e.g., the directions of arrows X and Y), or movable in three directions (e.g., the directions of arrows X, Y, and Z). In the case of two moving directions, the first direction need only be nonparallel to the second direction and is preferably orthogonal or substantially orthogonal to the second direction. In this case, a plane including the first direction and the second direction is preferably substantially parallel to the surface on which the tool placement portion 48 is arranged. In the case of three moving directions, the third direction need only intersect with a plane including the first direction and the second direction, for example, and is preferably orthogonal or substantially orthogonal to the plane including the first direction and the second direction. In the case where the laser moving unit is capable of moving the laser irradiation unit 53 in the direction substantially orthogonal to the surface on which the tool placement portion 48 is arranged, i.e., the bottom surface of the culture tool 100, the laser moving unit can adjust the spot diameter to be described below, for example. In this case, the laser moving unit also serves as a spot diameter adjustment unit to be described below, for example. In the present embodiment, the XY stage 51 is a known stage capable of moving an object at high speed and precisely along the directions of arrows X and Y via, for example, a linear motor carriage.

Preferably, the laser moving unit and the second imaging moving unit are capable of moving the laser irradiation unit 53 and the second imaging moving unit, respectively, in the first direction (e.g., the direction of arrow Y in FIG. 18A) in a plane substantially parallel to the surface on which the tool placement portion 48 is arranged, and the movement of the laser irradiation unit 53 by the laser moving unit in the first direction and the movement of the second imaging unit by the second imaging moving unit in the first direction are on the same straight line, as with the XY stage 51 according to the present embodiment. When the laser irradiation unit 53 and the second imaging unit move on the same straight line as described above, it is possible to reduce the number of times each unit is moved during processing such as, for example, capturing an image of the inside of the culture tool 100 by the second imaging unit and then processing the culture tool 100 by the laser irradiation unit 53, whereby the time required for the processing can be reduced. Further, as with the XY stage 51 according to the present embodiment, it is preferable that: the laser moving unit includes the carriage 511a on which the laser irradiation unit 53 is arranged and the moving path (rail) that is arranged along the first direction and on which the carriage 511a moves; the second imaging moving unit includes the carriage 511b on which the second imaging unit is arranged and the moving path (rail) that is arranged along the first direction and on which the carriage 511b moves; and the moving path of the laser moving unit and the moving path of the second imaging unit are the same. With such a structure, it is possible to further reduce the number of times each unit is moved during the processing such as the image capturing by the second imaging unit and the processing performed thereafter by the laser irradiation unit 53, whereby the time required for the processing can be further reduced.

Although the microscope 52 having the objective lenses 521a to 521c with three different magnifications is provided as the second imaging unit in the processing apparatus 400 of the present embodiment, the second imaging unit is not limited thereto and need only be capable of capturing images of the inside of the culture tool 100 placed in the tool placement portion 48. The second imaging unit is not limited to particular types of imaging units, and may be a known imaging unit such as a microscope or a camera, which may be used in combination with a solid-state imaging element (image sensor) such as a CCD or a complementary MOS (CMOS). The microscope may be an optical microscope such as a phase-contrast microscope or a fluorescence microscope. The microscope may have functions of both the phase-contrast microscope and the fluorescence microscope, for example. The second imaging unit is preferably capable of capturing images at a plurality of magnifications, but may be capable of capturing images at one magnification. As a specific example, when the second imaging unit is a microscope, the microscope preferably includes objective lenses with a plurality of magnifications (e.g., different magnifications). In the present embodiment, the magnifications of the objective lenses 521a to 521c are, for example, 2, 4, and 8 times, respectively. The second imaging unit may be capable of, for example, optical zooming or digital zooming. When the first imaging unit and the second imaging unit are included as in the processing apparatus 400 of the present embodiment, the magnification of the second imaging unit is preferably higher than the magnification of the first imaging unit because this allows capturing of clearer images of the inside of the culture tool 100.

In the processing apparatus 400 of the present embodiment, the laser irradiation unit 53 includes the laser source 531, the laser emission section 532, and the optical fiber 533. However, the laser irradiation unit 53 is not limited thereto and need only be capable of applying a laser to the culture tool 100 placed in the tool placement portion 48. The laser irradiation unit 53 may include the laser source 531, for example, and the laser source 531 may directly apply a laser to the culture tool 100. In the case where a laser from the laser source 531 is guided to the laser emission section 532, the laser may be guided using, instead of the optical fiber 533, a light guide unit such as a mirror or a micro electro mechanical system (MEMS). However, the optical fiber 533 is preferable because this allows the laser source 531 to be arranged at any desired position in the second region 5, and for example, by arranging the laser source 531 in a portion in which other units such as the laser moving unit, the second imaging unit, and the second imaging moving unit are not arranged and that does not overlap the movable ranges of the other units, the size of the processing apparatus 400 can be reduced, and the weight of the processing apparatus 400 can be reduced as compared with the case of using other light guide units.

The laser source 531 is, for example, a device that emits a continuous-wave laser or a pulsed laser. The laser source 531 may emit, for example, a high-frequency laser that has a long pulse width and approximates to a continuous wave. The output power of a laser emitted by the laser source 531 is not limited to particular values, and can be determined as appropriate according to, for example, the photothermal conversion molecules in the photothermal conversion layer 13. The wavelength of a laser emitted by the laser source 531 is not limited to particular values, and the laser may be, for example, a laser with a wavelength of 405 nm, 450 nm, 520 nm, 532 nm, or 808 nm, such as a visible-light laser or an infrared laser. When the culture tool 100 includes a laser absorption layer as described above, the laser source 531 generates, for example, a laser with a wavelength that can be absorbed by the laser absorption layer. As a specific example, the laser source 531 may be a continuous-wave diode laser having a maximum output power of 5 W and a wavelength in the vicinity of 405 nm.

When the laser irradiation unit 53 includes the laser emission section 532, it is preferable that the laser moving unit moves the laser emission section 532. When the laser moving unit moves the laser emission section 532 in the vertical direction (the direction of arrow Z in FIG. 18), the laser emission section 532 is preferably moved in such a manner that the laser emission aperture of the laser emission section 532 does not come into contact with the bottom surface of the processing chamber, preferably the bottom surface of the tool placement portion 48. As a specific example, the laser moving unit preferably moves the laser emission section 532 in such a manner that the laser emission aperture of the laser emission section 532 does not approach within 1 mm from the bottom surface of the tool placement portion 48. When the laser moving unit moves the laser emission section 532 within such a range, it is possible to prevent, for example, swaying of a solution such as a culture medium in the culture tool 100 placed in the tool placement portion 48 caused by the contact between the laser emission section 532 and the bottom surface of the tool placement portion 48.

In the present embodiment, the microscope 52 as the second imaging unit is arranged on the front side (the lower left side in FIG. 18), and the laser irradiation unit 53 is arranged on the back side (the upper right side in FIG. 18). However, the positional relationship between the second imaging unit and the laser irradiation unit 53 is not limited thereto, and the second imaging unit may be arranged on the back side and the laser irradiation unit 53 may be arranged on the front side, for example. In general, the second imaging unit such as a microscope has a larger volume than the laser irradiation unit 53. Thus, when the tool placement portion 48 is arranged on the front side in the first region 4, the size of the processing apparatus 400 can be reduced by arranging the second imaging unit on the back side and the laser irradiation unit 53 on the front side.

The processing apparatus 400 of the present embodiment may further include a spot diameter adjustment unit for adjusting the diameter of a spot formed in a portion to be irradiated with the laser in an object to be irradiated. The spot diameter means the beam diameter of a laser at a contact portion between the laser and the object to be irradiated. The spot diameter can be adjusted by, for example, switching at least one of a laser focusing lens and a collimator lens (collimation lens) of the laser irradiation unit 53 or changing the distance between the laser irradiation unit 53 and the object to be irradiated. In the former case, it is preferable that the laser irradiation unit 53 includes, for example, a plurality of lenses and that the spot diameter adjustment unit adjust the spot diameter through switching among the lenses. The plurality of lenses may be, for example, a plurality of focusing lenses, a plurality of collimator lenses, or a combination of at least one focusing lens and at least one collimator lens. The plurality of focusing lenses have focal lengths that differ from each other, for example. The plurality of collimator lenses have focal lengths that differ from each other, for example. Switching of the lenses may be performed, for example, manually or by a spot diameter adjustment control section to be described below. In the latter case, for example, a lens switching unit is provided, and switching of the lenses is performed by the lens switching unit. In the case where the spot diameter adjustment unit changes the distance, it is preferable that the spot diameter adjustment unit adjusts the spot diameter by adjusting the distance between the laser irradiation unit 53 and the object to be irradiated. The distance between the laser irradiation unit 53 and the object to be irradiated means, for example, the distance as measured in the direction substantially orthogonal to the surface on which the tool placement portion 48 is arranged, i.e., the bottom surface of the culture tool 100. In the case where the laser irradiation unit 53 includes the laser emission section 532, the distance between the laser irradiation unit 53 and the object to be irradiated means the distance between the laser emission section 532 and the object to be irradiated. The distance between the laser irradiation unit 53 and the object to be irradiated can be adjusted by, for example, the laser moving unit. As a specific example, by moving the laser irradiation unit 53 in the direction of arrow Z by the laser moving unit, the distance to the bottom of the culture tool 100 as the object to be irradiated can be adjusted. In the processing apparatus 400 of the present embodiment, the carriage 511a of the XY stage 51 as the laser moving unit can be moved up and down in the vertical direction (the direction of arrow Z). Thus, the laser moving unit in the present embodiment can also be referred to as, for example, a spot diameter adjustment unit. The spot diameter adjustment unit adjusts the spot diameter to, for example, make it smaller for processing in which a small spot diameter is preferable, for example. The spot diameter adjustment unit adjusts the spot diameter to make it larger for processing in which a large spot diameter is preferable, for example. The spot diameter is not limited to particular diameters, and can be set as appropriate according to the type of processing. When the processing apparatus 400 of the present embodiment includes the spot diameter adjustment unit, the spot diameter can be adjusted to an appropriate size at the time of processing the culture tool 100, thereby enabling rapid processing, for example. Moreover, since the spot diameter can be adjusted to an appropriate size, the cell adhesion region 11a in the culture tool 100 can be formed with high formability, for example.

When the processing apparatus 400 of the present embodiment includes the spot diameter adjustment unit, it is preferable that a control section to be described below includes a spot diameter adjustment control section for controlling the adjustment of the spot diameter by the spot diameter adjustment unit.

In the processing apparatus 400 of the present embodiment, it is preferable that movement of gas is reduced between the processing chamber and the second region 5. The movement of gas can be reduced by, for example, sealing the adjacent portion of the processing chamber to the second region 5 using the above-described sealing member such as a gasket or a sealant. By reducing the movement of gas as described above, it is possible to prevent, for example, dust contained in the gas from flowing into the processing chamber.

In the processing apparatus 400 of the present embodiment, the third region 6 includes a control unit 61 and a power supply section 62. As shown in FIG. 19, the control unit 61 includes structural components similar to those of a personal computer, a server computer, a workstation, or the like. As shown in FIG. 19, the control unit 61 includes a central processing unit (CPU) 61a, a main memory 61b, an auxiliary storage device 61c, a video codec 61d, an I/O interface 61e, and other components, and they are controlled by a controller (system controller, I/O controller, or the like) 61f and operate in cooperation with each other. Examples of the auxiliary storage device 61c include storage units such as a flash memory and a hard disk drive. The video codec 61d includes: a graphics processing unit (GPU) configured to generate a screen to be displayed based on a drawing instruction received from the CPU 61a and to transmit the screen signals to, for example, a display device or the like provided outside the processing apparatus 400; and a video memory for temporarily storing data concerning the screen and the image. The input-output (I/O) interface 61e is a device that is communicably connected to and controls the first XY stage 43a and the arm 43b (suction/discharge moving unit), the suction/discharge unit 44, the camera 80 (first imaging unit), the second XY stage 61 (laser moving unit and second imaging moving unit), the microscope 52 (second imaging unit), the laser irradiation unit 53, and the like. The I/O interface 61e may include a servo driver (servo controller). The I/O interface 61e may be connected to, for example, an input device provided outside the processing apparatus 400. Examples of the display device include monitors that output images (e.g., various image display devices such as a liquid crystal display (LCD) and a cathode ray tube (CRT) display). Examples of the input device 8 include pointing devices such as a touch panel, a trackpad, and a mouse, a keyboard, and a push button, each operable by an operator with his/her fingers.

The program executed by the control unit 61 is stored in the auxiliary storage device 61c. At the time of executing the program, the program is read into the main memory 61b and decoded by the CPU 61a. Then, the control unit 61 controls each member according to the program.

The control unit 61 in the present embodiment includes, in addition to the structural components of the control unit 22 in the second embodiment, an irradiation control section, a suction/discharge control section, a first imaging control section, and a second imaging control section. However, the irradiation control suction, the suction/discharge control suction, the first imaging control suction, and the second imaging control suction are optional structural components and may or may not be present. In the processing apparatus 400 of the present embodiment, the control unit 61 has the functions of the irradiation control section, the suction/discharge control section, the first imaging control section, the second imaging control section, and the like. Thus, it is not necessary to provide separate control units for the respective members, thereby allowing the size reduction of the processing apparatus. It is to be noted, however, that the present invention is not limited thereto. For example, in order to reduce the load on the control unit 61, each member may be provided with a control section, and the control unit 61 and the control sections of the respective members may work together to control the respective members. As specific examples, the laser emission and the like may be controlled by, for example, the control section provided in each member, and the movement of the laser irradiation unit 53 may be controlled by, for example, the control unit 61. The control unit 61 may be composed of one semiconductor element, may be a chip in which a plurality of semiconductor elements are formed in one package, or may include a plurality of semiconductor elements provided on a substrate.

In the present embodiment, the irradiation control section controls laser irradiation performed by the laser irradiation unit 53 and the movement of the laser emission section 532 of the laser irradiation unit 53 performed by the laser moving unit, namely, the XY stage 51 and the carriage 511. However, the laser control section may control either one of them.

In the present embodiment, the suction/discharge control section controls suction and discharge performed by the suction/discharge unit 44 and the movement of the suction/discharge unit 44 performed by the suction/discharge moving unit, namely, the XY stage 43a and the arm 43b. However, the suction/discharge control unit may control either one of them.

In the present embodiment, the first imaging control section controls image capturing of the inside of the processing chamber performed by the first imaging unit, namely, the camera 80.

In the present embodiment, the second imaging control section controls image capturing performed by the second imaging unit, namely, the microscope 52 and the movement of the microscope 52 performed by the second imaging moving unit, namely, the XY stage 51 and the carriage 511b. However, the second imaging control section may control either one of them.

The power supply section 62 is not limited to particular power supplies, and a known power supply can be used. The power supply section 62 supplies electric power to, for example, members (units) powered by electricity, such as the laser irradiation unit 53, the laser moving unit, the first imaging unit, the second imaging unit, the second imaging moving unit, the suction/discharge unit 44, the suction/discharge moving unit, the circulation unit 7, the illumination unit, the germicidal unit, and the control unit 61. Thus, the power supply section 62 is electrically connected to, for example, the members (units) powered by electricity. The power supply section 62 supplies electric power at a voltage of 100 V, for example. This allows the processing apparatus 400 to be used in a typical electric power environment, for example. In the processing apparatus 400 of the present embodiment, the power supply section 62 is responsible for the entire power supply. Thus, it is not necessary to provide separate power supply sections for the respective members, thereby allowing the size reduction and weight reduction of the processing apparatus 400, for example. It is to be noted, however, that the present invention is not limited thereto, and, for example, a dedicated power supply section may be provided for at least one of these units.

In the processing apparatus 400 of the present embodiment, a communication section (not shown) may be further provided in the third region 6. The communication section has a function of transmitting/receiving data to/from external devices such as a personal computer and a mobile communication device or a function of connecting to the Internet or the like through, for example, wired or wireless means. The communication section may be an existing communication module, for example. By providing the communication section as described above, it becomes possible to connect the processing apparatus 400 to external devices, and this allows the processing apparatus 400 to be operated from the outside or to receive data from the outside, for example. Also, it becomes possible to browse data stored in the processing apparatus 400 through connection to the processing apparatus 400 from the outside, for example.

Next, processing of a culture tool using the processing apparatus 400 of the present embodiment will be described with reference to an illustrative example.

First, the germicidal lamp 82 is turned off, and the illumination lamps 81a and 81b are turned on. The first imaging control section activates the camera 80 to start capturing of an image of the inside of the processing chamber. The image of the inside of the processing chamber captured by the camera 80 is output to the display device via, for example, the control unit 61. Next, the circulation unit 7 is activated to circulate the gas inside the processing chamber. Further, a user opens the door 42a of the opening 41a, and places a culture tool 100 in the tool placement portion 48. After placing the culture tool 100, the operator closes the door 42a of the opening 41a.

Next, the XY stage 51 and the carriage 511b are moved under the control of the second imaging control section, whereby the microscope 52 is moved to be located below the bottom surface of the culture tool 100. Also, the XY stage 43a is moved under the control of the suction/discharge control section, whereby the light source 45 is moved to be located above the upper surface of the culture tool 100, i.e., above the tool placement portion 48. Then, focusing of the microscope 52 is performed such that the microscope 52 is focused on the bottom surface 12a of the culture tool 100, and the distance to the culture tool 100 is measured. Focusing of the microscope 52 may be performed a plurality of times using, for example, the objective lenses 521a to 521c that differ from each other in magnification. The microscope 52 may capture images over time. In this case, the images captured over time by the microscope 52 may be, for example, phase-contrast microscope images captured by a phase-contrast microscope or fluorescence microscope images captured by a fluorescence microscope. The captured images are output to the display device via, for example, the control unit 61.

When the user inputs, for example, information on a user-specified irradiation region using the input device, the control unit 61 sets an irradiation region based on the input information on the irradiation region in the same manner as the control unit in the second embodiment. Subsequently, the control unit 61 controls the laser irradiation unit 53 based on the irradiation region such that the laser irradiation unit 53 applies the laser L to a region of the photothermal conversion layer 13 corresponding to the irradiation region.

Thereafter, the user opens the door 42a of the opening 41a and takes out the culture tool 100 from the tool placement portion 48. Thus, the culture tool 100 processed by the processing apparatus 400 of the present embodiment can be collected.

The processing apparatus 400 of the present embodiment can easily control a region to which cells can adhere in a cell culture tool including a cell culture base layer and a photothermal conversion layer in, for example, an aseptic condition or a clean space.

While the present invention has been described above with reference to exemplary embodiments, the present invention is by no means limited these embodiments. Various changes and modifications that may become apparent to those skilled in the art may be made in the configuration and specifics of the present invention without departing from the scope of the present invention.

This application claims priority from Japanese Patent Application No. 2020-044841 filed on Mar. 14, 2020. The entire disclosure of this Japanese patent application is incorporated herein by reference.

<Supplementary Notes>

Part or the whole of the exemplary embodiments and examples disclosed above can be described as in the following supplementary notes. It is to be noted, however, that the present invention is by no means limited thereto.

### (Supplementary Note 1)

A processing apparatus for a cell culture tool, including:

a laser irradiation unit capable of applying a laser to a photothermal conversion layer of a cell culture tool including a cell culture base layer and the photothermal conversion layer; and

a control unit for controlling the laser irradiation unit, wherein

the control unit includes a setting section and an irradiation control section,

the setting section sets an irradiation region to be irradiated with the laser in the cell culture tool, and

the irradiation control section controls the laser irradiation unit based on the irradiation region such that the laser irradiation unit apples the laser to a corresponding region of the photothermal conversion layer.

### (Supplementary Note 2)

The processing apparatus according to Supplementary Note 1, wherein

the control unit includes a dividing section,

the dividing section divides the irradiation region into segments based on an irradiation width of the laser, and

the irradiation control section controls the laser irradiation unit based on the respective segments of the irradiation region such that the laser irradiation unit applies the laser to corresponding regions of the photothermal conversion layer.

### (Supplementary Note 3)

The processing apparatus according to Supplementary Note 2, wherein

the laser irradiation unit includes a position acquisition section,

the position acquisition section acquires positions of endpoints of the respective segments of the irradiation region and associates the positions with the respective segments, and

the irradiation control section controls the laser irradiation unit based on the respective segments and the positions of the endpoints of the respective segments such that the laser irradiation unit applies the laser to the corresponding regions of the photothermal conversion layer by moving the laser in a direction from one endpoint toward the other endpoint in each segment.

### (Supplementary Note 4)

The processing apparatus according to Supplementary Note 3, wherein

the irradiation control section controls the laser irradiation unit based on the respective segments and the positions of the endpoints of the respective segments such that the laser irradiation unit applies the laser to the corresponding regions of the photothermal conversion layer by moving the laser in a direction from one endpoint toward the other endpoint in each segment,

a direction in which the laser is moved in a preceding segment is changed in a subsequent segment such that the laser is moved in a direction from the other endpoint toward the one endpoint of the preceding segment, and

the control in this manner is performed with respect to the entire irradiation region.

### (Supplementary Note 5)

The processing apparatus according to Supplementary Note 3 or 4, wherein

the position acquisition section acquires laser ON/OFF switching positions for the respective segments of the irradiation region, and

the irradiation control section controls the laser irradiation unit such that:

based on the respective segments and the positions of the endpoints of the respective segments, the laser irradiation unit applies the laser to the corresponding regions of the photothermal conversion layer by moving the laser in a direction from one endpoint toward the other endpoint in each segment; and

based on the laser ON/OFF switching positions, the laser is turned on or turned off.

### (Supplementary Note 6)

The processing apparatus according to any one of Supplementary Notes 2 to 5, wherein

the dividing section divides the irradiation region into approximately circular segments or spiral segments based on the irradiation width of the laser.

### (Supplementary Note 7)

The processing apparatus according to any one of Supplementary Notes 1 to 6, wherein

the control unit includes an acquisition section,

the acquisition section acquires irradiation region information in which the irradiation region is specified, and

the setting section sets the irradiation region based on the irradiation region information.

### (Supplementary Note 8)

The processing apparatus according to Supplementary Note 7, wherein

the irradiation region information includes an image in which the irradiation region is specified, and

the setting section sets the irradiation region based on a luminance value of the image in which the irradiation region is specified.

### (Supplementary Note 9)

The processing apparatus according to Supplementary Note 8, wherein

the irradiation region information includes information on a user-specified irradiation region, and

the setting section sets the irradiation region based on the information on the user-specified irradiation region.

### (Supplementary Note 10)

The processing apparatus according to any one of Supplementary Notes 1 to 6, wherein

the control unit includes an acquisition section,

the acquisition section acquires an image including the cell culture tool, and acquires irradiation region information in which the irradiation region is specified by extracting the irradiation region from the image, and

the setting section sets the irradiation region based on the irradiation region information.

### (Supplementary Note 11)

The processing apparatus according to any one of Supplementary Notes 1 to 6, wherein

the control unit includes an acquisition section,

the acquisition section identifies the cell culture tool in the image, and acquires irradiation region information associated with the cell culture tool based on the thus-acquired identification information for the cell culture tool, and

the setting section sets the irradiation region based on the irradiation region information.

### (Supplementary Note 12)

The processing apparatus according to any one of Supplementary Notes 1 to 6, wherein

the control unit includes an identification information acquisition section and an acquisition section,

the identification information acquisition section acquires identification information for the cell culture tool,

the acquisition section acquires irradiation region information associated with the cell culture tool based on the identification information for the cell culture tool, and

the setting section sets the irradiation region based on the irradiation region information.

### (Supplementary Note 13)

The processing apparatus according to any one of Supplementary Notes 1 to 12, further including a displacement measurement section, wherein

the displacement measurement section is capable of measuring a distance to the cell culture tool.

### (Supplementary Note 14)

The processing apparatus according to Supplementary Note 13, wherein

the control unit includes a displacement adjustment section, and

the displacement adjustment section adjusts a position of the laser irradiation unit based on the distance to the cell culture tool.

### (Supplementary Note 15)

The processing apparatus according to any one of Supplementary Notes 1 to 14, further including a first region, a second region, and a third region, wherein

the first region and the second region are arranged in succession,

the first region is a processing chamber for processing a cell culture tool,

the processing chamber can be closed from the outside of the processing chamber and includes a tool placement portion for placing the cell culture tool,

the second region includes the laser irradiation unit, and the laser irradiation unit can apply a laser to the cell culture tool placed in the tool placement portion,

the third region includes the control unit,

the tool placement portion is arranged in the processing chamber so as to be adjacent to the second region, and

an adjacent portion of the tool placement portion to the second region is capable of transmitting light.

### (Supplementary Note 16)

The processing apparatus according to Supplementary Note 15, wherein the processing chamber includes an opening and a door capable of opening and closing the opening.

### (Supplementary Note 17)

The processing apparatus according to Supplementary Note 16, wherein the door does not transmit light.

### (Supplementary Note 18)

The processing apparatus according to Supplementary Note 16 or 17, wherein

the processing chamber includes an opening for operations relating to processing of the cell culture tool in the processing chamber and an opening for enabling maintenance of the processing chamber, and

the opening for operations and the opening for enabling maintenance are provided at different positions in the processing chamber.

### (Supplementary Note 19)

The processing apparatus according to Supplementary Note 18, wherein the opening for operations has a smaller opening area than the opening for enabling maintenance.

### (Supplementary Note 20)

The processing apparatus according to Supplementary Note 18 or 19, wherein

the processing chamber further includes a germicidal unit capable of disinfecting the inside of the processing chamber, and

the germicidal unit is arranged on a side closer to the opening for operations in the processing chamber.

### (Supplementary Note 21)

The processing apparatus according to any one of Supplementary Notes 15 to 20, further including a circulation unit for circulating gas in the processing chamber, wherein

the circulation unit includes:
an intake section for sucking the gas inside the processing chamber;
a gas supply section for supplying part of the sucked gas into the processing chamber; and
an exhaust section for discharging the remainder of the sucked gas to the outside of the processing chamber.

### (Supplementary Note 22)

The processing apparatus according to Supplementary Note 21, wherein the exhaust section is arranged in an uppermost part of the processing apparatus.

### (Supplementary Note 23)

The processing apparatus according to Supplementary Note 21 or 22, wherein, in the processing apparatus according to any one of Supplementary Notes 16 to 20, the intake section is arranged near the opening of the processing chamber.

### (Supplementary Note 24)

The processing apparatus according to any one of Supplementary Notes 21 to 23, wherein

the processing chamber includes an outer wall and an inner wall, and

a circulation path that connects the intake section to the gas supply section and to the exhaust section is arranged between the outer wall and the inner wall.

### (Supplementary Note 25)

The processing apparatus according to any one of Supplementary Notes 15 to 24, wherein the processing chamber further includes an illumination unit capable of projecting light to the processing chamber.

### (Supplementary Note 26)

The processing apparatus according to any one of Supplementary Notes 15 to 25, wherein movement of gas is reduced between the processing chamber and the second region.

### (Supplementary Note 27)

The processing apparatus according to any one of Supplementary Notes 15 to 26, wherein

the processing chamber further includes a suction/discharge unit and a suction/discharge moving unit for moving the suction/discharge unit, and

the control unit includes a suction/discharge control section for controlling suction and discharge performed by the suction/discharge unit and movement of the suction/discharge unit performed by the suction/discharge moving unit.

### (Supplementary Note 28)

The processing apparatus according to Supplementary Note 27, wherein

the processing chamber includes a drainage container placement portion in which a drainage container for draining a liquid sucked by the suction/discharge unit can be placed, and

the tool placement portion and the drainage container placement portion are arranged along a moving direction of the suction/discharge moving unit in a plane substantially parallel to a surface on which the cell culture tool is placed.

### (Supplementary Note 29)

The processing apparatus according to Supplementary Note 27 or 28, wherein

the processing chamber includes:

a storage container placement portion in which a tip member storage container containing a tip member detachable from the suction/discharge unit can be placed, and

a tip member detachment unit for detaching the tip member from the suction/discharge unit.

### (Supplementary Note 30)

The processing apparatus according to any one of Supplementary Notes 15 to 29, wherein

the processing chamber includes a first imaging unit capable of capturing images of the inside of the processing chamber, and

the control unit includes a first imaging control section for controlling image capturing of the inside of the processing chamber by the first imaging unit.

### (Supplementary Note 31)

The processing apparatus according to any one of Supplementary Notes 15 to 30, wherein

the second region includes a second imaging unit capable of capturing images of the inside of the culture tool placed in the tool placement portion, and

the control unit includes a second imaging control section for controlling image capturing performed by the second imaging unit, and

the second imaging unit is capable of capturing images at a plurality of magnifications.

### (Supplementary Note 32)

The processing apparatus according to any one of Supplementary Notes 15 to 31, wherein

the laser irradiation unit includes a laser source and a laser emission section, and

the laser source is arranged in a portion of the second region in which other units are not arranged.

### (Supplementary Note 33)

The processing apparatus according to any one of Supplementary Notes 15 to 32, wherein

the second region includes:
a second imaging unit capable of capturing images of the cell culture tool placed in the tool placement portion;
a laser moving unit for moving the laser irradiation unit; and
a second imaging moving unit for moving the second imaging unit,

the control unit includes:
an irradiation control section for controlling laser irradiation performed by the laser irradiation unit and movement of the laser irradiation unit performed by the laser moving unit; and
a second imaging control section for controlling image capturing performed by the second imaging unit and movement of the second imaging unit performed by the second imaging moving unit,

the laser moving unit is capable of moving the laser irradiation unit in a first direction in a plane substantially parallel to the surface on which the tool placement portion is arranged,

the second imaging moving unit is capable of moving the second imaging unit in the first direction in the plane substantially parallel to the surface on which the tool placement portion is arranged, and

movement of the laser irradiation unit in the first direction by the laser moving unit and movement of the second imaging unit in the first direction by the second imaging moving unit are on the same straight line.

### (Supplementary Note 34)

The processing apparatus according to Supplementary Note 33, wherein

the laser moving unit includes a carriage on which the laser irradiation unit is arranged and a moving path that is arranged along the first direction and on which the carriage moves,

the second imaging moving unit includes a carriage on which the second imaging unit is arranged and a moving path that is arranged along the first direction and on which the carriage moves, and

the moving path for the laser moving unit and the moving path for the second imaging moving unit are the same.

### (Supplementary Note 35)

The processing apparatus according to Supplementary Note 33 or 34, wherein the laser moving unit is capable of moving the laser irradiation unit further in a direction substantially orthogonal to the surface on which the tool placement portion is arranged.

### (Supplementary Note 36)

The processing apparatus according to any one of Supplementary Notes 33 to 35, wherein the laser moving unit is capable of moving the laser irradiation unit in a second direction substantially orthogonal to the first direction in the plane substantially parallel to the surface on which the tool placement portion is arranged.

### (Supplementary Note 37)

The processing apparatus according to any one of Supplementary Notes 15 to 36, wherein

a bottom surface of the tool placement portion includes a recess for placing the cell culture tool,

the recess includes a protruding portion that protrudes inward at its end on a side closer to the second region, and

a side surface of the recess has an inversely tapered shape that narrows from the inside toward the outside of the processing chamber.

### (Supplementary Note 38)

The processing apparatus according to any one of Supplementary Notes 1 to 37, further including a monitoring (imaging) unit capable of monitoring (imaging) the inside of the cell culture tool.

### INDUSTRIAL APPLICABILITY

The processing apparatus according to the present invention can control a region to which cells can adhere in a cell culture tool including a cell culture base layer and a photothermal conversion layer. Therefore, the present invention is very useful, for example, in the fields of regenerative medicine and drug discovery.

1. A processing apparatus for a cell culture tool, comprising:
a laser irradiation unit configured to apply a laser to a photothermal conversion layer of a cell culture tool including a cell culture base layer and the photothermal conversion layer; and
a control unit for controlling the laser irradiation unit, the control unit comprising at least one processor, wherein
the processor is configured to
set an irradiation region to be irradiated with the laser in the cell culture tool, and
control the laser irradiation unit based on the irradiation region such that the laser irradiation unit apples the laser to a corresponding region of the photothermal conversion layer.

2. The processing apparatus according to claim 1, wherein
the processor is configured to
divide the irradiation region into segments based on an irradiation width of the laser, and
control the laser irradiation unit based on the respective segments of the irradiation region such that the laser irradiation unit applies the laser to corresponding regions of the photothermal conversion layer.

3. The processing apparatus according to claim 2, wherein
the laser irradiation unit comprises a position acquisition section,
the position acquisition section acquires positions of endpoints of the respective segments of the irradiation region and associates the positions with the respective segments, and
the processor is configured to control the laser irradiation unit based on the respective segments and the positions of the endpoints of the respective segments such that the laser irradiation unit applies the laser to the corresponding regions of the photothermal conversion layer by moving the laser in a direction from one endpoint toward the other endpoint in each segment.

4. The processing apparatus according to claim 3, wherein
the processor is configured to control the laser irradiation unit based on the respective segments and the positions of the endpoints of the respective segments such that the laser irradiation unit applies the laser to the corresponding regions of the photothermal conversion layer by moving the laser in a direction from one endpoint toward the other endpoint in each segment,
a direction in which the laser is moved in a preceding segment is changed in a subsequent segment such that the laser is moved in a direction from the other endpoint toward the one endpoint of the preceding segment, and
the control in this manner is performed with respect to the entire irradiation region.

5. The processing apparatus according to claim 3, wherein
the position acquisition section acquires laser ON/OFF switching positions for the respective segments of the irradiation region, and
the processor is configured to control the laser irradiation unit such that:
based on the respective segments and the positions of the endpoints of the respective segments, the laser irradiation unit applies the laser to the corresponding regions of the photothermal conversion layer by moving the laser in a direction from one endpoint toward the other endpoint in each segment; and
based on the laser ON/OFF switching positions, the laser is turned on or turned off.

6. The processing apparatus according to claim 2, wherein
the processor is configured to divide the irradiation region into approximately circular segments or spiral segments based on the irradiation width of the laser.

7. The processing apparatus according to claim 1, wherein
the processor is configured to acquire irradiation region information in which the irradiation region is specified, and
set the irradiation region based on the irradiation region information.

8. The processing apparatus according to claim 7, wherein
the irradiation region information comprises an image in which the irradiation region is specified, and
the processor is configured to set the irradiation region based on a luminance value of the image in which the irradiation region is specified.

9. The processing apparatus according to claim 8, wherein
the irradiation region information comprises information on a user-specified irradiation region, and
the processor is configured to set the irradiation region based on the information on the user-specified irradiation region.

10. The processing apparatus according to claim 1, wherein
the processor is configured to acquire an image including the cell culture tool, and acquires irradiation region information in which the irradiation region is specified by extracting the irradiation region from the image, and
set the irradiation region based on the irradiation region information.

11. The processing apparatus according to claim 1, wherein
the processor is configured to identify the cell culture tool in the image, and acquires irradiation region information associated with the cell culture tool based on the thus-acquired identification information for the cell culture tool, and
set the irradiation region based on the irradiation region information.

12. The processing apparatus according to claim 1, wherein
the processor is configured to acquire identification information for the cell culture tool,
acquire irradiation region information associated with the cell culture tool based on the identification information for the cell culture tool, and
set the irradiation region based on the irradiation region information.

13. The processing apparatus according to claim 1, further comprising a displacement measurement section, wherein
the displacement measurement section is configured to measure a distance to the cell culture tool.

14. The processing apparatus according to claim 13, wherein
the processor is configured to adjust a position of the laser irradiation unit based on the distance to the cell culture tool.

15. The processing apparatus according to claim 1, further comprising a first region, a second region, and a third region, wherein
the first region and the second region are arranged in succession,
the first region is a processing chamber for processing a cell culture tool,
the processing chamber can be closed from the outside of the processing chamber and comprises a tool placement portion for placing the cell culture tool,
the second region comprises the laser irradiation unit, and the laser irradiation unit can apply a laser to the cell culture tool placed in the tool placement portion,
the third region comprises the control unit,
the tool placement portion is arranged in the processing chamber so as to be adjacent to the second region, and
an adjacent portion of the tool placement portion to the second region is capable of transmitting light.
